(19) United States
(12) Patent Application Publication
(10) Pub. No.: US 2026/0165745 A1
(43) Pub. Date: Jun. 18, 2026

(54) BONE ANCHOR ASSEMBLY WITH BI-SPHERIC SHANK HEAD AND INTEGRAL, TWIST-INTO-POSITION AND DOWNWARDLY DISPLACEABLE COLLET INSERT

(71) Applicant: Roger P. Jackson, Prairie Village, KS (US)

(72) Inventors: Roger P. Jackson, Prairie Village, KS (US); James L. Surber, Kansas City, KS (US); Nathaniel D. Ginzton, Boise, ID (US)

(21) Appl. No.: 19/419,864

(22) Filed: Dec. 15, 2025

Related U.S. Application Data
(60) Provisional application No. 63/734,072, filed on Dec. 14, 2024.

(51) Int. Cl. A61B 17/70; A61B 17/86

(52) U.S. Cl. CPC A61B 17/7032; A61B 17/7005; A61B 17/7037; A61B 17/863

(57) ABSTRACT

A bone anchor assembly includes a bone anchor having a shank head at a proximal end and an anchor portion at a distal end, and a receiver having a receiver channel for receiving a rod, a central bore with opposed inner engagement grooves below a closure mating structure, and a spherical seating surface adjacent a bottom opening. The assembly also includes a cap retainer positionable within a lower portion of the central bore and having plurality of retainer collet fingers configured to resiliently expand to capture the shank head within the receiver. The assembly further includes a collet insert having an insert channel for engaging the rod, opposite outer engagement ridges, and a plurality of insert collet fingers configured to resiliently expand to capture the cap retainer. Prior to uploading the shank head through the bottom opening, the collet insert is maintained in an initial vertical position within the central bore with the cap retainer being uploaded into collet insert so that a lower opening of the cap retainer is spaced above the bottom opening of the receiver.

### CROSS-REFERENCE TO RELATED APPLICATIONS

This application claims the benefit of U.S. Provisional Application No. 63/734,072, filed Dec. 14, 2024, which is incorporated by reference in its entirety herein and for all purposes.

### FIELD

The present disclosure relates generally to modular spinal implant assemblies utilizing bi-spheric shank heads that are configured for connection with a collection or array of pivoting and non-pivoting but axially rotatable (e.g., monoaxial) receiver sub-assemblies having different functionalities, and their use in surgery involving vertebral body stabilizations with spinal fixation systems.

### BACKGROUND

Spinal implants in general, and bone anchors or screws in particular, are used in many types of spinal surgery in order to secure various implants to vertebrae along the spinal column for the purposes of treating spinal disorders, such as degenerative conditions and deformities, and also for stabilizing and/or adjusting spinal alignment. A common mechanism for providing vertebral support is to implant the bone screws into certain bones which then, in turn, support a longitudinal structure such as an elongate rod, or are supported by such a rod. Although both closed-ended and open-ended spinal implants, such as bone screws and hooks, are known, the open-ended spinal implants can be particularly well suited for connections to rods and connector arms because such rods or arms do not need to be passed through a closed bore, but rather can be laid or urged into an open channel within the head or receiver of such a screw, hook, or connector. For example, open-ended bone screws generally comprise an anchor portion, such as a threaded shank, connected to a head or receiver having a pair of upwardly-projecting branches or arms which form a yoke that defines a slot or channel configured to receive the rod. The slot or channel could have different shapes, such as, a U-shape or a square shape. Moreover, the threaded shanks of the bone screws can also be replaced with hooks or other types of bone anchors or connectors to form a variety of different types of spinal implants, also having open ends for receiving rods or portions of other structures, and wherein such implants can facilitate surgical techniques performed with different spinal fixation systems.

Early bone screws or anchors used in spinal surgery generally had a yoke-shaped ‘head’ that was integrally formed or “fixed” with the threaded shank, and therefore immovable. Because the fixed head could not be moved relative to the shank, these fixed bone screws needed to be favorably positioned in the spine; otherwise, the elongate rod would need to be bent in order for it to be placed within the rod-receiving channels of a linear series of adjacent bone screws, due to their alignment. Given the highly curved shape of the spines of some patients, however, this is sometimes very difficult or impossible to do. Therefore, polyaxial (i.e., multiplanar), uni-planar (i.e., monoplanar), and/or translatable pivotal bone screws or bone anchor assemblies, were developed and are now commonly preferred. Open-ended polyaxial bone screw assemblies typically allow for pivoting and rotation of the connected but completely separate yoke-shaped receiver or receiver sub-assembly about an enlarged spherical ‘head’ or upper capture portion of the threaded shank or bone anchor in one or more planes, until a desired rotational and pivotal position of the receiver is achieved relative to the shank. This can be accomplished by manipulating the position of the receiver relative to the shank during a final stage of a medical procedure when the elongate rod or other longitudinal connecting member is inserted into the receiver or receiver sub-assembly, followed by a locking set screw, a plug, a closure, or other type of hard locking mechanism known in the art.

It is understood that spinal fixation systems generally include a variety of components that require some assembly, such as the various types of bone anchors, the rods or connector arms, and the closures or plugs with the receivers or receiver sub-assemblies, with each component having specific features with respect to structure and function. Moreover, the receiver sub-assemblies can further include components in addition to the receiver itself, such as pressure inserts, wave washers, separate retainers, and other components of different types that are operable to connect these receiver sub-assemblies with the heads of the bone anchors. The pressure inserts, rings, retainers, and other components can be pre-assembled together within the receivers to form the receiver sub-assemblies that are ready for further assemblage with the bone anchors, and eventually with the rods or connector arms and the closures or plugs.

Some designs provide for the threaded shanks or other types of bone anchors to be bottom loaded into the receiver sub-assemblies. With bottom loaded bone anchor assemblies, for example, some designs known in the art require a retaining component (e.g., the collet portion of an insert or a separate retainer) to hold the shank within the receiver, with the receiver having a bottom opening large enough to allow for the head or upper capture portion of the threaded shank or bone anchor to be uploaded into the central bore or cavity of the receiver. Other types of bottom loaded bone anchor assemblies do not include the retaining component, however, and instead include a receiver having a lower portion with a bottom opening that is configured to directly threadably mate with the head or upper capture portion of the shank that can be configured as a threaded spherical head to provide for polyaxial or multiplanar motion.

Further to the above, bottom loaded bone anchor assemblies can also be fully assembled by the spinal company or distributor before being shipped to a hospital, so as to help with inventory management, or can be shipped as a modular array of multiple separate and different shanks and a fewer number of pre-assembled receiver sub-assemblies that can then be fully assembled, for example, at the hospital or surgical center during a surgery, thereby saving costs. Additionally, the modular spinal implants can be fully assembled at the hospital either before insertion into the patient, or after the threaded shank or bone anchor has been inserted into the patient, either by a surgeon, with or without robotic assistance, or also directly by a robot. The different techniques or approaches for the insertion and assembly of the modular parts of the bone anchor assemblies can be described as ex-vivo and in-vivo, respectively.

### SUMMARY

The present disclosure is generally directed to modular spinal fixation systems with bone anchors comprising a certain type of common or universal shank head configured to connect with a wide array of receiver sub-assemblies having different functionalities to form pivotal and non-pivotal bone anchor assemblies with different capabilities. To that purpose, one embodiment of the present disclosure comprises a spinal fixation system for securing an elongate rod to a spine of a patient.

The spinal fixation system includes a plurality of bone anchors, with each bone anchor having a longitudinal axis, a bi-spheric shank head at a proximal end devoid of outer parallel planar side surfaces, an anchor portion opposite the shank head configured for fixation to the bone, and a neck portion extending between the shank head and the anchor portion. Each bi-spheric shank head includes an upper partial spherical portion comprising an upper spherical surface having a first diameter extending downward from an upper end, out and around the hemisphere plane of the upper spherical surface, to a circular inner edge of upward-facing shelf surface of a lower shelf or ledge structure that is spaced below the hemisphere plane, and a lower partial spherical portion comprising a lower spherical surface having a second diameter that is greater than the first diameter and which extends downward from the circular outer edge of the upward-facing shelf surface toward the neck portion that connects the bi-spheric shank head to the anchor portion. In one aspect the upward-facing shelf surface is an annular planar surface extending perpendicular to the longitudinal axis of the bone anchor.

The spinal fixation system also includes an array of receiver sub-assemblies, with each receiver sub-assembly including a receiver with a base portion that defines a lower section of a central bore centered around a vertical centerline axis and communicating with a bottom of the receiver through a bottom opening, and an upper portion having a channel configured to receive the elongate rod describe above. The central bore includes a seating surface adjacent or proximate the bottom opening, and extends upward through the channel to a top of the receiver. Each receiver sub-assembly also includes one of a multiplanar pivoting retaining structure (also known as a cap retainer), a monoplanar pivoting retaining structure or cap retainer, or a non-pivoting or monoaxial retaining structure or cap retainer positioned therein and configured to slidably engage the seating surface after capturing the upper partial spherical portion of a bi-spheric shank head upon its uploading through the bottom opening of the receiver.

Each receiver sub-assembly further includes a collet insert that is initially positionable within the central bore above the retaining structure. The collet insert has an upper surface configured to engage the elongate rod and an expandable lower collet portion configured to receive and hold the cap retainer in a centralized and stabilized position that is spaced above the bottom opening of the receiver when the receiver sub-assembly is in a shipping state configuration. After the uploading of the bi-spheric shank head of the bone anchor through the bottom opening, and its capture within the cap retainer and the lower collet portion, the collet insert is downwardly deployable with tooling (or with an elongate rod and closure) until the cap retainer/bi-spheric shank head enter into engagement with the lower seating surface of the receiver, thereby securing the bone anchor to the receiver sub-assembly and forming a bone anchor assembly. As described in more detail below, in one aspect the completed bone anchor assembly can have a pre-lock friction fit configuration.

In particular, once the bi-spheric shank head of the bone anchor has been captured by the retaining structure or cap retainer of one of the retainer sub-assemblies, ex-vivo or in-vivo, and the collet insert has been downwardly deployed to form the bone anchor assembly, the bone anchor is further configured to have frictional axial independent rotation with respect to the receiver sub-assembly, together with one of multiplanar motion or monoplanar frictional pivoting motion with respect to the receiver sub-assembly for the pivotal bone anchor assemblies.

Another embodiment of the present disclosure can comprise a bone anchor assembly including a bone anchor having a shank head at a proximal end and an anchor portion at a distal end, and a receiver having a receiver channel for receiving a rod, a central bore with opposed inner engagement grooves below a closure mating structure, and a spherical seating surface adjacent a bottom opening. The bone anchor assembly also includes a cap retainer positionable within a lower portion of the central bore and having plurality of retainer collet fingers configured to resiliently expand to capture the shank head within the receiver. The bone anchor assembly further includes a collet insert having an insert channel for engaging the rod, opposite outer engagement ridges, and a lower collet portion defined by a plurality of insert collet fingers configured to resiliently expand to capture the cap retainer. Prior to uploading the shank head through the bottom opening, the collet insert is maintained in an initial vertical position within the central bore with the cap retainer being uploaded into the lower collet portion so that a lower opening of the cap retainer is spaced above the bottom opening of the receiver.

At least one additional embodiment of the present disclosure includes non-pivoting receiver sub-assemblies in which the upper ends of the retaining structures and the lower ends of the collet inserts are configured to form a stepped cylindrical joint when engaged together, with the retaining structures being rotatable about the vertical centerline axis of the receiver relative to the collet inserts prior to hard locking the receiver assemblies to the shank heads.

Other additional embodiments of the present disclosure will be better understood upon review of the detailed description set forth below taken in conjunction with the accompanying drawing figures, which are briefly described as follows.

### BRIEF DESCRIPTION OF THE DRAWINGS

FIG. 1 is a partially-sectioned front perspective view of a multi-component spinal fixation system showing four major types of receiver sub-assemblies and a universal bone anchor having a bi-spheric universal shank head, with each receiver sub-assembly having different functionalities and being attachable to the defined common geometry on the upper capture portion of the bone anchor, in accordance with a representative embodiment of the present disclosure.

FIG. 2 is a perspective view of the bi-spheric shank head or upper capture portion of the universal bone anchor of FIG. 1.

FIG. 3 is a cross-sectional view of the universal bone anchor of FIG. 1.

FIG. 4 is a cross-sectional view of the bi-spheric shank head or upper capture portion of the universal bone anchor of FIG. 1.

FIG. 5 is a perspective view of the multiplanar cap retainer of the multiplanar bone anchor assembly of FIG. 1(b).

FIG. 6 is a cross-sectional perspective view of the multiplanar cap retainer of FIG. 6.

FIG. 7 is a partially-sectioned perspective view of the multiplanar cap retainer and bi-spheric shank head of the multiplanar bone anchor assembly of FIG. 1(b) showing the multiplanar shank head sub-assembly.

FIG. 8 is a partially-sectioned front view of the multiplanar shank head sub-assembly of FIG. 7.

FIG. 9 is a partially-sectioned perspective view of the multiplanar bone anchor assembly of FIG. 1(b) in an articulated position or configuration, in accordance with the representative embodiment of the multi-component spinal fixation system shown in FIG. 1.

FIG. 10 is an exploded perspective view of the multiplanar bone anchor assembly shown in FIG. 9.

FIG. 11 is a perspective view of the multiplanar receiver of the multiplanar bone anchor assembly of FIGS. 9-10.

FIG. 12 is a top view of the multiplanar receiver of FIG. 11.

FIG. 13 is a cross-sectional perspective view of the multiplanar receiver of FIG. 11.

FIG. 14 is another cross-sectional perspective view of the multiplanar receiver of FIG. 11.

FIG. 15 is a perspective view of the multiplanar collet insert of the multiplanar bone anchor assembly of FIGS. 9-10.

FIG. 16 is a bottom perspective view of the multiplanar collet insert of FIG. 15.

FIG. 17 is a side view of the multiplanar collet insert of FIG. 15.

FIG. 18 is a cross-sectional side view of the multiplanar collet insert of FIG. 15.

FIG. 19 is a top perspective view of the closure of the multiplanar bone anchor assembly of FIGS. 9-10.

FIG. 20 is a bottom perspective view of the closure of FIG. 19.

FIG. 21 is an exploded partially-sectioned perspective view of the components of the multiplanar receiver sub-assembly of FIG. 10 prior to their pre-assembly into a shipping configuration.

FIG. 22 is a partially-sectioned front view of the multiplanar receiver of FIG. 21 with the multiplanar cap retainer being downloaded through the rod channel of the multiplanar receiver.

FIG. 23 is a partially-sectioned front view of the multiplanar receiver of FIG. 21 with the seated multiplanar cap retainer contacting the spherical seating surface of the multiplanar receiver.

FIG. 24 is a partially-sectioned front view of the multiplanar receiver of FIG. 21, together with the seated multiplanar cap retainer and with the multiplanar collet insert being downloaded through the rod channel of the multiplanar receiver.

FIG. 25 is a partially-sectioned front view of the multiplanar receiver of FIG. 21, together with the seated multiplanar cap retainer and with the multiplanar collet insert being further downloaded into the internal cavity of the multiplanar receiver.

FIG. 26 is a partially-sectioned front view of the multiplanar receiver of FIG. 21, together with the seated multiplanar cap retainer and the downloaded and partially-rotated multiplanar collet insert.

FIG. 27 is a partially-sectioned front view of the multiplanar receiver of FIG. 21, together with the seated multiplanar cap retainer and the fully-rotated multiplanar collet insert.

FIG. 28 is a partially-sectioned front view of the multiplanar receiver of FIG. 21, together with the multiplanar collet insert being fully rotated therein and the multiplanar cap retainer being uploaded into the lower collet portion of the multiplanar collet insert to form the pre-assembled multiplanar receiver sub-assembly in the shipping state configuration.

FIG. 29 is a partially-sectioned front perspective view of the multiplanar receiver sub-assembly of FIG. 28.

FIG. 30 is a partially-sectioned perspective view of the multiplanar receiver sub-assembly of FIGS. 28-29 positioned above the bi-spheric shank head of the universal bone anchor.

FIG. 31 is partially-sectioned front view of the multiplanar receiver sub-assembly moving downward until the bi-spheric shank head engages the multiplanar cap retainer that is suspended within the internal cavity of the multiplanar receiver by the lower collet portion of the multiplanar collet insert.

FIG. 32 is partially-sectioned front view of the multiplanar receiver sub-assembly as the bi-spheric shank head continues to drive upward as it expands the multiplanar cap retainer within the lower collet portion of the multiplanar collet insert until reaching the maximum expansion of the multiplanar cap retainer.

FIG. 33 is a partially-sectioned front view of the multiplanar receiver sub-assembly as the bi-spheric shank head continues to drive upward until the bi-spheric shank head is completely captured by the multiplanar cap retainer to form the multiplanar shank head sub-assembly secured within the multiplanar receiver sub-assembly.

FIG. 34 is a partially-sectioned front view of the multiplanar receiver sub-assembly after the multiplanar collet insert has been downwardly deployed to push the multiplanar shank head sub-assembly back downward against the seating surface of the multiplanar receiver to establish the initial configuration of the multiplanar bone anchor assembly in a pre-lock friction fit.

FIG. 35 is a partially-sectioned perspective view of the assembled multiplanar bone anchor assembly with the pre-lock friction fit and the multiplanar shank head sub-assembly being pivoted to an articulated position.

FIG. 36 is a partially-sectioned front view of the multiplanar bone anchor assembly, fully assembled with the elongate rod and closure and with the bone anchor in an articulated position relative to the multiplanar receiver.

FIG. 37 is a close-up partially-sectioned front view of a portion of the fully-assembled multiplanar bone anchor assembly of FIG. 36, with the bone anchor in a non-articulated position relative to the multiplanar receiver.

FIG. 38 is a close-up partially-sectioned front view of the multiplanar bone anchor assembly, fully assembled with a 5.5 mm elongate rod and closure.

FIG. 39 is a close-up partially-sectioned front view of the multiplanar bone anchor assembly, fully assembled with a 6.0 mm elongate rod and closure.

FIG. 40 is an exploded perspective view of the multiplanar IL (“Independent Lock”) bone anchor assembly of FIG. 1(c), in accordance with the representative embodiment of the multi-component spinal fixation system shown in FIG. 1.

FIG. 41 is a perspective view of the IL collet insert of the multiplanar IL bone anchor assembly of FIG. 40.

FIG. 42 is a bottom perspective view of the IL collet insert of FIG. 41.

FIG. 43 is a front view of the IL collet insert of FIG. 41.

FIG. 44 is a side view of the IL collet insert of FIG. 41.

FIG. 45 is a top perspective view of the two-piece closure of the multiplanar IL bone anchor assembly of FIG. 40.

FIG. 46 is a bottom perspective view of the two-piece closure of FIG. 45.

FIG. 47 is a top view of the outer ring of the two-piece closure of FIG. 45.

FIG. 48 is a cross-sectional perspective view of the outer ring of the two-piece closure of FIG. 45.

FIG. 49 is a perspective view of the center set screw of the two-piece closure of FIG. 45.

FIG. 50 is a cross-sectional perspective view of the center set screw of the two-piece closure of FIG. 45.

FIG. 51 is an exploded partially-sectioned perspective view of the components of the multiplanar IL receiver sub-assembly of FIG. 40 prior to their pre-assembly into a shipping configuration.

FIG. 52 is a partially-sectioned perspective view of the multiplanar receiver of FIG. 51, together with the seated multiplanar cap retainer and with the IL collet insert being downloaded through the rod channel of the receiver.

FIG. 53 is a partially-sectioned perspective view of the multiplanar receiver of FIG. 51, together with the seated multiplanar cap retainer and with the IL collet insert being further downloaded into the internal cavity of the receiver.

FIG. 54 is a partially-sectioned front view of the multiplanar receiver of FIG. 51, together with the seated multiplanar cap retainer and the downloaded and partially-rotated IL collet insert.

FIG. 55 is a partially-sectioned front view of the multiplanar receiver of FIG. 51, together with the seated multiplanar cap retainer and the fully-rotated IL collet insert.

FIG. 56 is a partially-sectioned front view of the multiplanar receiver of FIG. 51, together with the IL collet insert being fully rotated therein and the multiplanar cap retainer being uploaded into the lower collet portion of the IL collet insert to form the pre-assembled multiplanar IL receiver sub-assembly in the shipping state configuration.

FIG. 57 is a partially-sectioned front perspective view of the multiplanar IL receiver sub-assembly of FIG. 56.

FIG. 58 is a partially-sectioned front view of the multiplanar IL receiver sub-assembly of FIGS. 56-57 positioned above the bi-spheric shank head of the universal bone anchor.

FIG. 59 is partially-sectioned front view of the multiplanar IL receiver sub-assembly of FIG. 58 moving downward until the bi-spheric shank head engages the cap retainer that is suspended within the internal cavity of multiplanar receiver by the lower collet portion of the IL collet insert.

FIG. 60 is partially-sectioned front view of the multiplanar IL receiver sub-assembly as the bi-spheric shank head continues to drive upward as it expands the multiplanar cap retainer within the lower collet portion of the IL collet insert until reaching the maximum expansion of the cap retainer.

FIG. 61 is a partially-sectioned front view of the multiplanar IL receiver sub-assembly as the bi-spheric shank head continues to drive upward until the bi-spheric shank head is completely captured by the multiplanar cap retainer to form the multiplanar shank head sub-assembly secured within the receiver sub-assembly.

FIG. 62 is a partially-sectioned front view of the multiplanar IL receiver sub-assembly after the IL collet insert has been downwardly deployed to push the multiplanar shank head sub-assembly back downward against the seating surface of the multiplanar receiver to establish the initial configuration of the multiplanar IL bone anchor assembly in a pre-lock friction fit.

FIG. 63 is a partially-sectioned perspective view of the assembled multiplanar IL bone anchor assembly with the pre-lock friction fit and the multiplanar shank head sub-assembly being pivoted to an articulated position.

FIG. 64 is a partially-sectioned perspective view of the multiplanar IL bone anchor assembly together with the two-piece closure and with the bone anchor in an articulated position relative to the receiver.

FIG. 65 is a partially-sectioned front view of the multiplanar IL bone anchor assembly, fully assembled with a 6.0 mm elongate rod and closure.

FIG. 66 is a close-up partially-sectioned front view of the multiplanar IL bone anchor assembly, fully assembled with a 5.5 mm elongate rod and closure.

FIG. 67 is an exploded perspective view of the monoplanar bone anchor assembly of FIG. 1(d), in accordance with the representative embodiment of the multi-component spinal fixation system shown in FIG. 1.

FIG. 68 is a cross-sectional perspective view of the monoplanar receiver of the monoplanar bone anchor assembly of FIG. 67.

FIG. 69 is another cross-sectional perspective view of the monoplanar receiver of FIG. 68.

FIG. 70 is a perspective view of the monoplanar cap retainer of the monoplanar bone anchor assembly of FIG. 67.

FIG. 71 is a cross-sectional perspective view of the monoplanar cap retainer of FIG. 70.

FIG. 72 is a side view of the monoplanar cap retainer of FIG. 70.

FIG. 73 is a close-up cross-sectional perspective view of a portion of the monoplanar cap retainer of FIG. 70.

FIG. 74 is a perspective view of the monoplanar collet insert of the monoplanar bone anchor assembly of FIG. 67.

FIG. 75 is a bottom perspective view of the monoplanar collet insert of FIG. 74.

FIG. 76 is a cross-sectional side view of the monoplanar collet insert of FIG. 74.

FIG. 77 is a bottom view of the monoplanar collet insert of FIG. 74.

FIG. 78 is an exploded partially-sectioned perspective view of the components of the monoplanar receiver sub-assembly of FIG. 67 prior to their pre-assembly into a shipping configuration.

FIG. 79 is a partially-sectioned perspective view of the monoplanar receiver of FIG. 78 with the monoplanar cap retainer being downloaded through the rod channel of the monoplanar receiver.

FIG. 80 is a partially-sectioned perspective view of the monoplanar receiver of FIG. 78 with the seated monoplanar cap retainer contacting the spherical seating surface of the monoplanar receiver.

FIG. 81 is a partially-sectioned perspective view of the monoplanar receiver of FIG. 78, together with the seated monoplanar cap retainer and with the monoplanar collet insert being downloaded through the rod channel of the monoplanar receiver.

FIG. 82 is a partially-sectioned front view of the monoplanar receiver, cap retainer and collet insert of FIG. 81.

FIG. 83 is a partially-sectioned front view of the monoplanar receiver of FIG. 78, together with the seated monoplanar cap retainer and the partially-downloaded and partially-rotated monoplanar collet insert.

FIG. 84 is a partially-sectioned front view of the monoplanar receiver of FIG. 78, together with the seated monoplanar cap retainer and the partially-downloaded, fully-rotated monoplanar collet insert.

FIG. 85 is a partially-sectioned front view of the receiver of FIG. 78, together with the monoplanar collet insert being fully downloaded therein and the monoplanar cap retainer being uploaded into the lower collet portion of the collet insert to form the pre-assembled monoplanar receiver sub-assembly in the shipping state configuration.

FIG. 86 is an isolated side view of the monoplanar cap retainer and the monoplanar collet insert of FIG. 85, with the monoplanar cap retainer being uploaded into the lower collet portion of the monoplanar collet insert.

FIG. 87 is a partially-sectioned front view of the monoplanar receiver sub-assembly of FIG. 85 positioned above the bi-spheric shank head of the universal bone anchor.

FIG. 88 is partially-sectioned front view of the monoplanar receiver sub-assembly moving downward until the bi-spheric shank head engages the cap retainer that is suspended within the internal cavity of the monoplanar receiver by the lower collet portion of the monoplanar collet insert.

FIG. 89 is partially-sectioned front view of the monoplanar receiver sub-assembly as the bi-spheric shank head continues to drive upward as it expands the monoplanar cap retainer within the lower collet portion of the monoplanar collet insert until reaching the maximum expansion of the monoplanar cap retainer.

FIG. 90 is a partially-sectioned front view of the monoplanar receiver sub-assembly as the bi-spheric shank head continues to drive upward until the bi-spheric shank head is completely captured by the monoplanar cap retainer to form the monoplanar shank head sub-assembly secured within the monoplanar receiver sub-assembly.

FIG. 91 is a partially-sectioned front view of the monoplanar receiver sub-assembly after the monoplanar collet insert has been downwardly deployed to push the monoplanar shank head sub-assembly back downward against the seating surface of the monoplanar receiver to establish the initial configuration of the monoplanar bone anchor assembly in a pre-lock friction fit.

FIG. 92 is a partially-sectioned perspective view of the assembled monoplanar bone anchor assembly with the pre-lock friction fit and the monoplanar shank head sub-assembly being pivoted to an articulated position.

FIG. 93 is a partially-sectioned front view of monoplanar bone anchor assembly, fully assembled with the elongate rod and closure and with the bone anchor in a non-articulated position relative to the monoplanar receiver.

FIG. 94 is an exploded perspective view of the monoaxial bone anchor assembly of FIG. 1(e), in accordance with the representative embodiment of the multi-component spinal fixation system shown in FIG. 1.

FIG. 95 is a cross-sectional perspective view of the receiver of the monoaxial bone anchor assembly of FIG. 94.

FIG. 96 is another cross-sectional perspective view of the receiver of FIG. 95.

FIG. 97 is a perspective view of the monoaxial collet insert of the monoaxial bone anchor assembly of FIG. 94.

FIG. 98 is a bottom perspective view of the monoaxial collet insert of FIG. 97.

FIG. 99 is a cross-sectional side view of the monoaxial collet insert of FIG. 97.

FIG. 100 is a bottom view of the monoaxial collet insert of FIG. 97.

FIG. 101 is a perspective view of the monoaxial cap retainer of the monoaxial bone anchor assembly of FIG. 94.

FIG. 102 is a cross-sectional view of the monoaxial cap retainer of FIG. 101.

FIG. 103 is an exploded perspective view of the components of the monoaxial receiver sub-assembly of FIG. 94 prior to their pre-assembly into a shipping configuration.

FIG. 104 is a partially-sectioned perspective view of the receiver of FIG. 103 with the monoaxial cap retainer being downloaded through the rod channel of the receiver.

FIG. 105 is a partially-sectioned perspective view of the receiver of FIG. 103 with the seated monoaxial cap retainer contacting the spherical seating surface of the receiver and with the monoaxial collet insert being downloaded through the rod channel of the receiver.

FIG. 106 is a partially-sectioned front view of the receiver of FIG. 103, together with the seated monoaxial cap retainer and with the monoaxial collet insert being further downloaded into the internal cavity of the receiver.

FIG. 107 is a partially-sectioned front view of the receiver of FIG. 103, together with the seated monoaxial cap retainer and the downloaded and partially-rotated monoaxial collet insert.

FIG. 108 is a partially-sectioned front view of the receiver of FIG. 103, together with the seated monoaxial cap retainer and the fully-rotated monoaxial collet insert.

FIG. 109 is a partially-sectioned front view of the receiver of FIG. 103, together with the monoaxial collet insert being fully rotated therein and the monoaxial cap retainer being uploaded into the lower collet portion of the monoaxial collet insert to form the pre-assembled monoaxial receiver sub-assembly in the shipping state configuration.

FIG. 110 is a partially-sectioned bottom perspective view of the monoaxial receiver sub-assembly of FIG. 109.

FIG. 111 is a partially-sectioned perspective view of the monoaxial receiver sub-assembly of FIGS. 109-110 positioned above the bi-spheric shank head of the universal bone anchor.

FIG. 112 is partially-sectioned front view of the monoaxial receiver sub-assembly of FIG. 111 moving downward until the bi-spheric shank head engages the monoaxial cap retainer that is suspended within the internal cavity of the r receiver by the lower collet portion of the monoaxial collet insert.

FIG. 113 is partially-sectioned front view of the monoaxial receiver sub-assembly as the bi-spheric shank head continues to drive upward as it expands the monoaxial cap retainer within the lower collet portion of the monoaxial collet insert until reaching the maximum expansion of the monoaxial cap retainer.

FIG. 114 is a partially-sectioned front view of the monoaxial receiver sub-assembly as the bi-spheric shank head continues to drive upward until the bi-spheric shank head is completely captured by the monoaxial cap retainer to form the monoaxial shank head sub-assembly secured within the monoaxial receiver sub-assembly.

FIG. 115 is a partially-sectioned front view of the monoaxial receiver sub-assembly after the monoaxial collet insert has been downwardly deployed to push the monoaxial shank head sub-assembly back downward against the seating surface of the receiver to establish the initial configuration of the monoaxial bone anchor assembly in a pre-lock friction fit.

FIG. 116 is a partially-sectioned perspective view of the monoaxial bone anchor assembly of FIG. 115, fully assembled with the elongate rod and closure and with the bone anchor in a non-articulated position relative to the receiver.

FIG. 117 is a close-up partially-sectioned front view of a portion of the fully-assembled monoaxial bone anchor assembly of FIG. 116.

FIG. 118 is a partially-sectioned perspective view of a multiplanar bone anchor assembly in an articulated position or configuration, in accordance with another representative embodiment of the present disclosure.

FIG. 119 is an exploded perspective view of the multiplanar bone anchor assembly shown in FIG. 118.

FIG. 120 is a perspective view of the multiplanar cap retainer of the multiplanar bone anchor assembly of FIGS. 118-119.

FIG. 121 is a closeup perspective view of the multiplanar cap retainer of FIG. 120.

FIG. 122 is a perspective view of the multiplanar collet insert of the multiplanar bone anchor assembly of FIGS. 118-119.

FIG. 123 is a cross-sectional side view of the multiplanar collet insert of FIG. 122.

FIG. 124 is a partially-sectioned perspective view of the multiplanar receiver of FIGS. 118-119, together with the multiplanar collet insert being fully rotated therein and the multiplanar cap retainer being uploaded into the lower collet portion of the multiplanar collet insert to form the pre-assembled multiplanar receiver sub-assembly in the shipping state configuration.

FIG. 125 is a partially-sectioned perspective view of the multiplanar bone anchor assembly of FIGS. 118-119, fully assembled with the elongate rod and closure and with the bone anchor in an articulated position relative to the multiplanar receiver.

### DETAILED DESCRIPTION OF EXEMPLARY EMBODIMENTS

The following description, in conjunction with the accompanying drawings, is provided as an enabling teaching of bone anchors having a representative type of ‘universal’ shank head configured to cooperate with separate retaining structures that, in turn, have been pre-assembled together with collet inserts into receivers to form receiver sub-assemblies with different functionalities, and with the bi-spheric shank heads being bottom-loaded into the pre-assembled receiver sub-assemblies. As described below, the representative type of universal shank head or capture portion of a bone anchor illustrated herein is a bi-spheric shank head or capture structure comprising an upper partial spherical portion of lesser diameter that extends below a hemisphere plane, and a lower partial spherical portion of greater diameter that begins at a lower offset plane that is spaced below the hemisphere plane to extend downward and merge with the neck of the shank body, and with an upward-facing shelf or annular ledge extending between the upper partial spherical portion and the lower partial spherical portion.

The bone anchors are generally configured for use with a collection or array of complementary pivotal and non-pivotal receiver sub-assemblies in a spinal fixation system. In particular, the collection can include different types of receiver sub-assemblies that can be coupled to the bi-spheric shank heads of the bone anchors to form bone anchor assemblies having different and specialized modes of movement, degrees of freedom, or modalities (with the terms ‘mode’, ‘modality’, and ‘multi-modal’, etc., being used herein to describe the way in which something moves), including but not limited to pivoting and non-pivoting but axially rotatable (e.g. monoaxial) movement of the receiver sub-assembly relative to a shank or bone anchor that is further configured for implantation into the bone of a patient. The description also includes one or more methods for assembling and employing the bone anchors with the multi-modal collection of receiver sub-assemblies. As described below, individual bone anchor assemblies, systems, and/or methods of assembly and/or use of the present disclosure for this representative type of universal shank head can provide significant advantages and benefits over other pivotal and/or non-pivotal bone anchors and spinal fixation systems known in the art due to, in one aspect, the degree of versatility and adaptability provided by the shank head universality (i.e. all of the shank heads having a common geometry that is connectable with each type of receiver sub-assembly that has its own predetermined combination of degrees of freedom and operational functionalities). The recited advantages are not meant to be limiting in any way, however, as one skilled in the art will appreciate that other advantages and benefits may also be realized upon practicing the present disclosure.

Furthermore, those skilled in the relevant art will recognize that changes can be made to the disclosed embodiments for shank head universality, beyond those described, while still obtaining the beneficial results. It will also be understood and appreciated that some of the advantages and benefits of the described embodiment for the invention can be obtained by selecting some of the features (e.g., the structures or components) of the disclosed receiver sub-assemblies without utilizing other features, and that features from one sub-assembly embodiment may be interchanged or combined with features from other sub-assemblies in any appropriate combination. For example, any individual feature or collective features of method embodiments may be applied to apparatus, product or system embodiments, and vice versa. Likewise, structural elements or functional features from one embodiment may also be combined with or replaced by structural elements or functional features from one or more additional embodiments in any suitable manner. Those who work in the art will therefore recognize that many modifications and adaptations to the representative embodiments described herein are possible and may even be desirable in certain circumstances, and are to be considered part of the present disclosure. Thus, it will be appreciated that the present disclosure is provided as an illustration of the principles for the representative modular spinal fixation system incorporating the bi-spheric shank head that are shown and discussed therein, since the scope of each invention disclosed herein is to be defined by their respective claims.

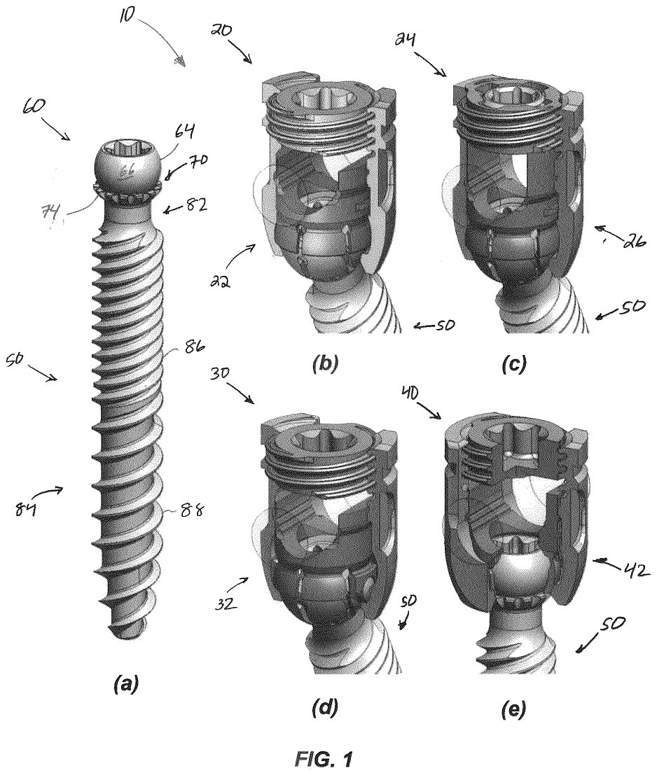

Referring now in more detail to the drawing figures, wherein like parts are identified with like reference numerals throughout the several views, FIG. 1 illustrates a modular spinal fixation system 10 having bone anchors (a.k.a. bone attachment structures such as screws, hooks, shanks, and other known anchor components) attached to longitudinal connecting members (such as rods, cords, connectors, and other known longitudinal connecting members) with bi-spheric shank heads 60 that can be bottom loaded into receiver sub-assemblies (i.e. housings or heads), and wherein the receiver sub-assemblies 22, 26, 32, 42 and at least some of their associated internal components can pivot and/or rotate axially in different selected directions relative to their bone anchors.

More specifically, receiver sub-assemblies that are configured to provide the fully assembled bone anchors 20, 24, 30, 40 with different modes of movement or degrees of freedom, such as multiplanar pivotal movement, monoplanar pivotal movement, and monoaxial movement (non-pivotal but axial rotatable), together with a variety of operational functionalities such as pre-lock friction fit with tool deployment of the collet insert, pre-lock friction fit without tool deployment, provisional independent locking, open top receivers, closed top receivers, and the like, can be pre-assembled with their internal components into receiver sub-assemblies 22, 26, 32, 42 that are configured to be snapped onto or otherwise connected to the bi-spheric shank head 60 or upper end capture structure, of one or more shanks or bone anchors 50 (which may or may not be cannulated).

The spinal fixation system 10 shown in FIG. 1 is directed toward eliminating or at least improving upon shortcomings of the prior art through the introduction of a bone anchor, such as the shank 50 shown in FIG. 1(a), having an upper end capture structure comprising the bi-spherical “universal” shank head, or bi-spheric shank head 60, with both modularity and bone debris clearance capabilities, and which is inherently free of flat side surfaces. In particular, the bi-spheric shank head 60 of the present disclosure is configured to be cleared of bone debris and soft tissue simultaneous with the process or motion of being “snapped” into, or otherwise connected, and captured by either a multiplanar pivotal and independently axially rotatable receiver sub-assembly 22, 26, a monoplanar pivotal and independently axially rotatable receiver sub-assembly 32, an independently axially rotatable but non-pivotal receiver sub-assembly 42, or any other type of receiver sub-assembly having an alternative mode of movement.

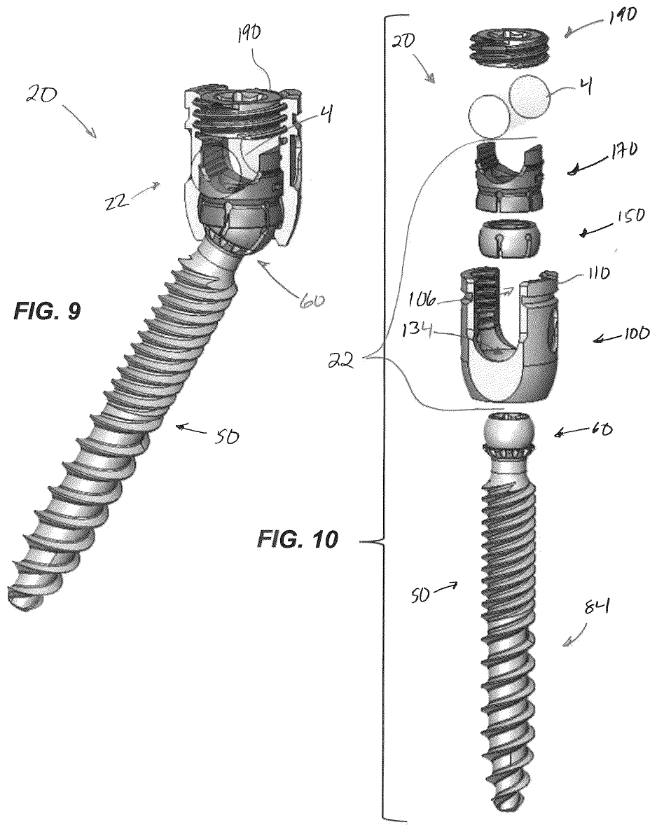

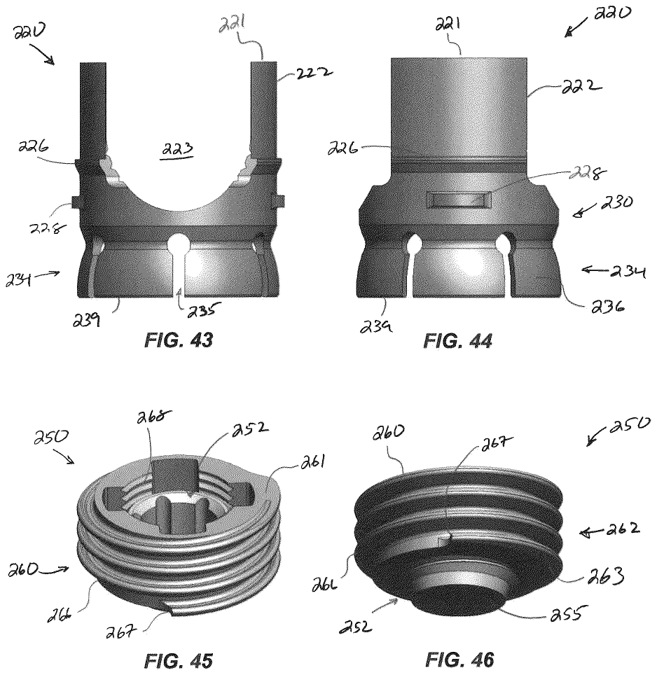

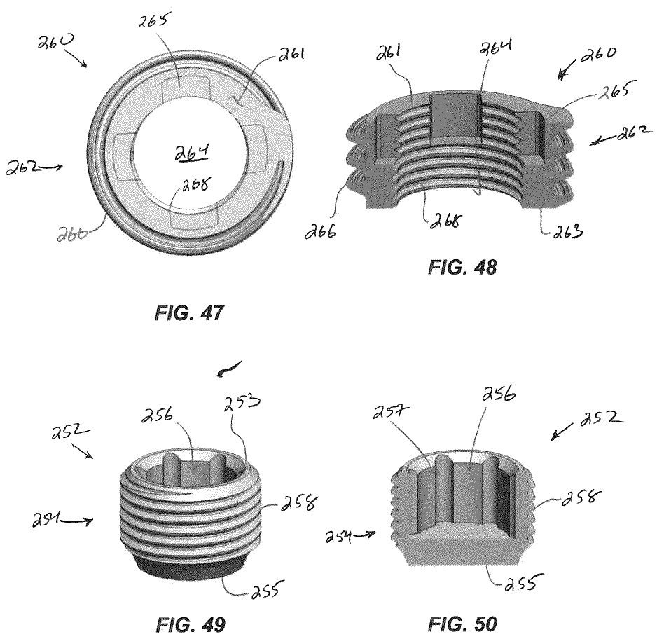

With reference to FIG. 1(b) and 1(c), the representative embodiments of the multiplanar pivotal and axially rotatable receiver sub-assemblies 22, 26 with bone debris clearance can be combined with a bone screw 50 having the bi-spheric shank head 60 to form multiplanar bone anchor assemblies 20, 24 further described in reference to FIGS. 9-66. The multiplanar bone anchor assemblies 20, 24 can include components having features or aspects configured to provide for pre-lock frictional pivotal motion of the bone anchor relative 50 to the receiver sub-assembly 22, 26 around a 360-degree range, and also to provide for pre-lock frictional axial rotation relative to a longitudinal axis of the bone anchor around a 360-degree range, and is hereinafter interchangeably referred to as a polyaxial, multi-axial, or ‘multiplanar’ bone anchor assembly.

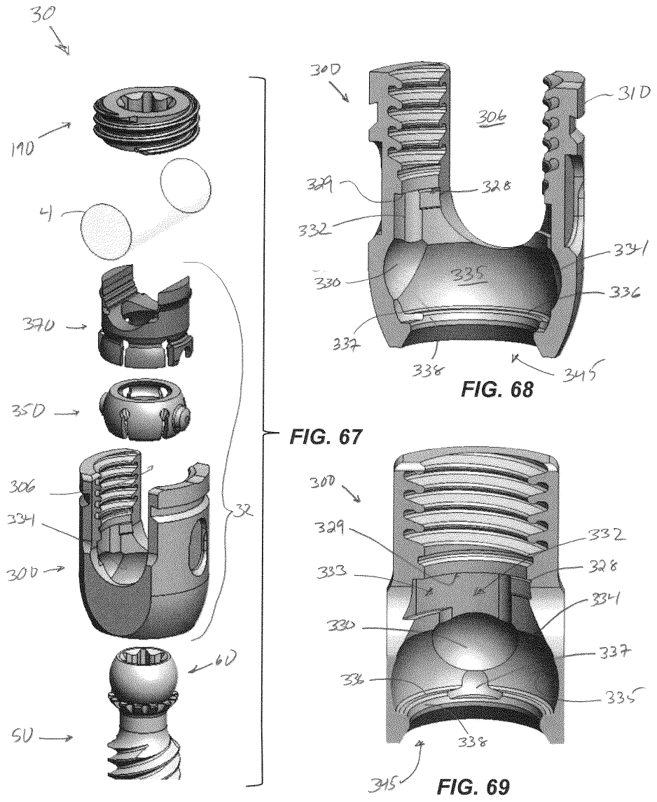

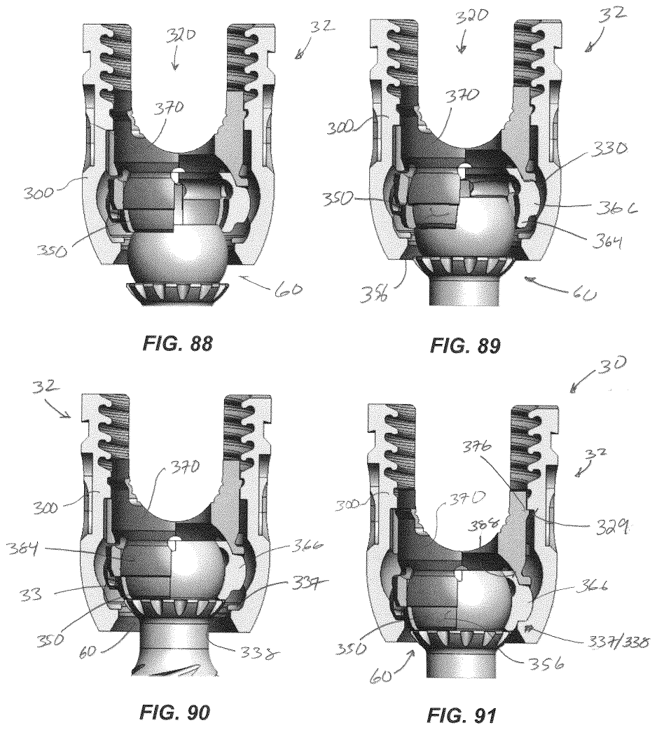

Similarly, the representative embodiment of the monoplanar pivotal and independently axially rotatable receiver sub-assembly 32 shown in FIG. 1(d) can be combined with a bone screw 50 having the bi-spheric shank head 60 to form a monoplanar bone anchor assembly 30 further described in reference to FIGS. 67-93. The monoplanar pivotal bone anchor assembly 30 can include alternative components having features or aspects configured to limit the pre-lock frictional pivotal motion of the bone anchor relative to the receiver sub-assembly 32 (or vice versa) to a single plane (i.e. sagittal, medial-lateral) while still providing for pre-lock frictional axial rotation around a 360-degree range, and is hereinafter interchangeably referred to as a uni-planar or ‘monoplanar’ bone anchor assembly 30. As shown in the drawings, the bi-spheric shank head 60 can be included into this monoplanar functionality without the use of parallel flat or planar side surfaces formed into the outer surfaces of the bi-spheric shank head.

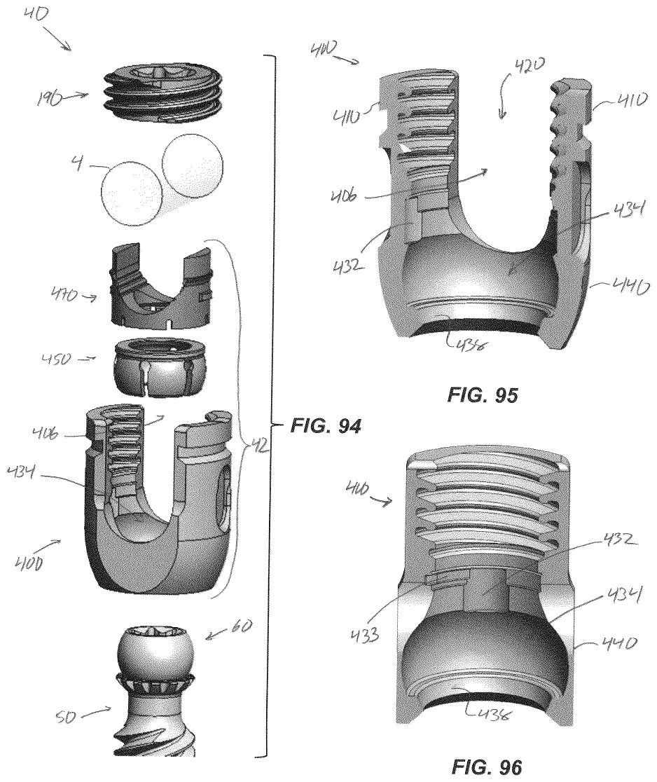

Likewise, the representative embodiment of the non-pivotal but axially rotatable receiver sub-assembly 42 shown in FIG. 1(e) can be combined with the same bi-spheric shank head 60, or upper end capture portion geometry, to form a monoaxial bone anchor assembly 40 further described in reference to FIGS. 94-117. The monoaxial bone anchor assembly 40 can also include alternative components having features or aspects configured to prevent or inhibit pivotal motion of the bone anchor 50 relative to the receiver sub-assembly 42 (or vice versa) with some possible limited toggle, while still providing for pre-lock frictional axial rotation around a 360-degree range, and is hereinafter interchangeably referred to as a non-pivotal, fixed, or ‘monoaxial’ bone anchor assembly 40. Again, as shown in the drawings, the bi-spheric shank head 60 can be included into this non-pivotal monoaxial functionality without the use of parallel flat or planar side surfaces formed into the outer surfaces of the bi-spheric shank head.

Thus, regardless of the type, degree or amount of pivotal motion, the three major modes of movement or modality embodiments of the bone anchor assembly 20, 24, 30, 40 are together configured to provide the multi-modal modular spinal fixation system 10 wherein the bone anchor 50 can axially rotate around its longitudinal or spin axis relative to the receiver sub-assembly 22, 26, 32, 42 (or vice versa) at least prior to locking the bone anchor assembly 20, 28, 630, 40 with the closure in the final locked position and with at least some degree of a pre-lock friction fit. It will be appreciated that this feature can allow for the rotatable implantation, or screwing in, of only the anchor portion 84 of a pre-assembled bone anchor assembly 20, 24, 30, 40 to a desired depth in the bone of a patient without rotation of its respective receiver sub-assembly 22, 26, 32, 42, thereby allowing the receiver sub-assembly to be secured by separate tooling, or maintained in a desired alignment, throughout the rotatable implantation of the bone anchor. This feature can also allow for the height of the receiver sub-assembly 22, 26, 32, 42 above the bone, or the length of the anchor portion 84 of the bone anchor 50 that is implanted in the bone, to be more precisely controlled and independently adjusted, and wherein more aggressive thread forms having larger pitches for faster insertions with fewer rotations can also be utilized, especially with robot assisted surgeries. In addition, the geometry of the upper end capture portion 60 of the screw 50 can further provide for a very strong and secure connection with a driving tool for navigated manual or robotic assisted screw insertions, or even direct robotic screw insertions.

It will be further appreciated that the bone anchors 50 can be connected with their respective pivoting or non-pivoting receiver sub-assemblies 22, 26, 32, 42 either before or after being affixed to the bony anatomy of a patient. In many embodiments, for example, the receiver sub-assemblies 22, 26, 32, 42 can be pre-assembled at a factory or manufacturing facility, and then further assembled with the bone anchor 50 into the fully-assembled bone anchor assemblies 20, 24, 30, 40 before being shipped to a spine company for inventory and/or storage, or to a hospital or surgery center for insertion into a patient. However, in other embodiments the pre-assembled receiver sub-assemblies can be shipped separately, prior to engagement with the bone anchor 50, to the spine company, hospital or surgery center. In this case the configuration of the separate pre-assembled receiver sub-assemblies may be defined as the “shipping state” configuration or condition.

Furthermore, the bone anchor assembly or spinal fixation system 10 may also be considered “modular” in the sense that any particular receiver sub-assembly 22, 26, 32, 42, in the shipping state condition, can be coupled with any one of a variety of bone anchors 50 having anchor portions of different size, length, type, and/or thread patterns, but with all of the bone anchors 50 having the same universal capture structure, such as the bi-spherical capture structure 60, at their upper ends. It will also be appreciated that a receiver sub-assembly 22, 26, 32, 42 in the shipping state condition can be assembled with the bone anchor 50 at the hospital or surgery center either before insertion into the patient, or after the threaded shank or bone anchor 50 has been inserted into the patient (such as directly by a surgeon or with robotic assistance). For instance, in some cases it may be desirable to implant or attach the shanks or bone anchors 50 into or on the spine of the patient independent of their larger and somewhat bulky receiver sub-assemblies, and decide later on in the surgical procedure where each of the receiver sub-assemblies 22, 26, 32, 42 should be placed and utilized on the implanted spinal construct. This type of multi-modal modular capability can be advantageous for both midline and pedicle screw placement trajectories into the vertebral bodies and to provide for enhanced procedural solutions in certain cases, including robotic assisted surgeries. As known to one of skill in the art, the different techniques or approaches for the insertion and assembly of the modular parts of the bone anchor assembly can be described as ex-vivo and in-vivo or in-situ, respectively.

### Bi-Spheric Universal Bone Anchor

Referring now in more detail to the drawing figures, specifically FIG. 1(a) and 2-4, the bone anchor 50 of the spinal fixation system 10 (including but not limited to the shank shown in the drawings) includes the bi-spheric shank head or capture structure 60 at an upper or proximal end 51, and a body 80 extending distally from the bi-spheric shank head 60 with an attachment or anchor portion 84 at a distal end 99 configured for fixation to the bone of a patient. The body of the shank 80 can be integral with the bi-spheric shank head 60 and can include a neck portion or neck 82 that extends between the bi-spheric shank head 60 and the anchor portion 84. In one aspect the neck 82 can have a cross-sectional diameter that is less than both the diameter(s) of the bi-spheric shank head 60 and the cross-sectional diameter of the anchor portion 84 immediately below the neck 82, and can be configured to pivot against an inner edge of the lower opening of the receiver of a bone anchor assembly 22, 28, 32, 42 so as to provide an increased angle of articulation between the receiver and the bone anchor 50. As shown, the anchor portion 84 can be a threaded anchor portion with one or more bone engagement threads, such as a full length dual-lead thread form 88 extending the length of the body of the shank 80 from the distal tip 96 to the neck 82, and a partial length dual-lead thread form 86 beginning at an intermediate location and extending along an upper portion of the shank body 80 to the neck 82.

The bi-spheric shank head 60 at the proximal end 52 of the shank 50 generally comprises an upper partial spherical portion 64 defining an upper spherical surface 66 that extends above and below a hemisphere plane 64 of the bi-spheric shank head 60, and a lower partial spherical portion 74 defining a lower spherical surface 76 that begins at a lower offset plane 73 that is spaced below the hemisphere plane 65 to extend downward and merge with the neck 82 of the shank body 80. A lower upward-facing shelf or annular ledge 70 extends between the upper inner partial spherical portion 64 and the lower partial spherical portion 74, and can be considered the portion of the lower partial spherical portion 74 that extends radially outward beyond the upper spherical surface 66. As shown in the drawings, in one aspect the annular lower ledge 70 can define an upward-facing planar ledge surface 72 that extends perpendicular to the longitudinal axis 51 of the shank along the lower offset plane 73 between the upper and lower partial spherical portions. It is foreseen, nevertheless, that the lower ledge may not extend along the lower offset plane and may instead intersect the lower offset plane and the upper edge of the lower partial spherical portion at an acute angle, thereby defining a generally upward-facing ledge surface that is frusto-conical rather than planar, whether extending upwardly and outwardly, or downwardly and outwardly, from the upper partial spherical portion 64 to the lower partial spherical portion 74.

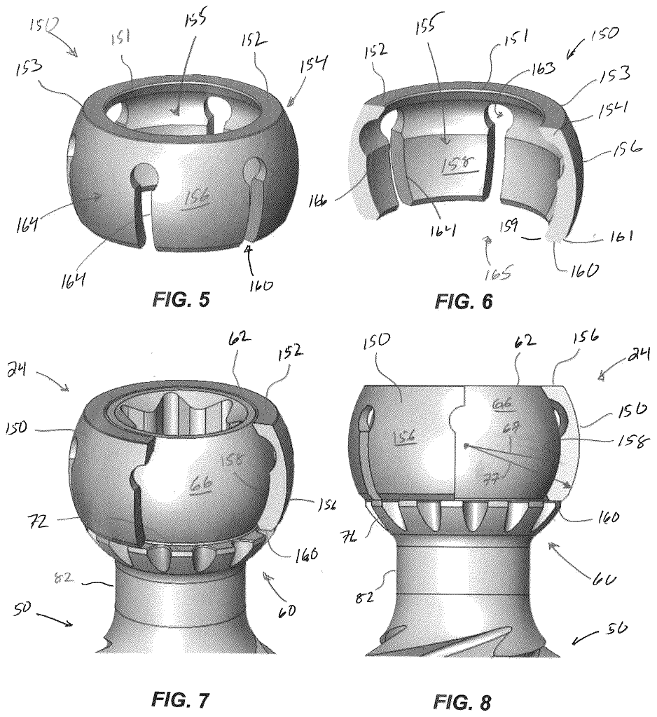

A detailed discussion of the structure and features of the bone anchor 50 and its universal capture portion 60 is provided in co-owned U.S. Pat. No. 12,414,801, filed Nov. 3, 2023, which is incorporated by reference in its entirety herein and for all purposes. Accordingly, a detailed discussion of the additional structures and features of the bone anchor 50 and its bi-spheric shank head 60, as well as the bi-spheric shank head's connection with a cap retainer (such as the multiplanar cap retainer 150 shown in FIGS. 5-8) and additional interactions with other components of the different receiver sub-assemblies 22, 26, 32, 42, will not be repeated herein.

### Multiplanar Bone Anchor Assembly

FIG. 9 is partially-sectioned perspective view of one representative embodiment of the multiplanar bone anchor assembly 20 illustrated in FIG. 1(b), with the multiplanar receiver sub-assembly 22 and an elongate rod 4 being connected to the bi-spheric shank head 60 of the bone anchor 50 with a single-piece closure 190, and with the bone anchor 50 being pivoted and locked at an angle with respect to the multiplanar receiver sub-assembly 22. FIG. 10 is an exploded perspective view of the same multiplanar bone anchor assembly 20 and rod 4.

With continued reference to FIG. 10, the multiplanar receiver sub-assembly 22 generally includes a multiplanar receiver 100 or housing that can be initially pivotably secured to the bi-spheric shank head 60 with a number of separate internal components that have been pre-assembled into a central bore and rod channel 106 of the multiplanar receiver 100 to form the multiplanar receiver sub-assembly 22. These internal components can include, but are not limited to, a pivoting or articulating multiplanar cap retainer 150 that can be positioned in the internal cavity 134 or lower portion of the central bore, and which attaches to the bi-spheric shank head 60 (see FIGS. 7-8) to pivotably couple the shank 50 to the receiver sub-assembly 22. The receiver sub-assembly 22 further includes a multiplanar collet insert 170, also known as a compression element, which can be positioned above the cap retainer 150 in a middle portion of the central bore where the central bore intersects with the rod channel, and is operable to engage with the cap retainer 150 below and to be engaged by the elongate rod 4 from above. In one aspect the collet insert 170 can be downwardly-displaceable with a tool or tooling from an upper shipping-state position to a lower friction-fit position after the bi-spheric shank head 60 has been uploaded into the receiver sub-assembly, so as to establish a non-floppy pre-lock friction fit configuration prior to final assembly with the elongate rod and the closure.

After the elongate rod 4 has been positioned within a lower portion of the rod channel and into engagement with the collet insert 170, a closure 190 can be threadably or otherwise secured into an upper portion of the central bore or rod channel to apply pressure to an upper surface of the rod 4, such as by direct contact, thereby locking both the elongate rod 4 and the multiplanar bone anchor assembly 20 into a final locked configuration or position, such as that shown in FIG. 9. It is foreseen that the collet insert 170 can also be downwardly-displaceable from the shipping state position to the friction fit and/or locked position simultaneous with the placement of the rod and threaded installation of the closure.

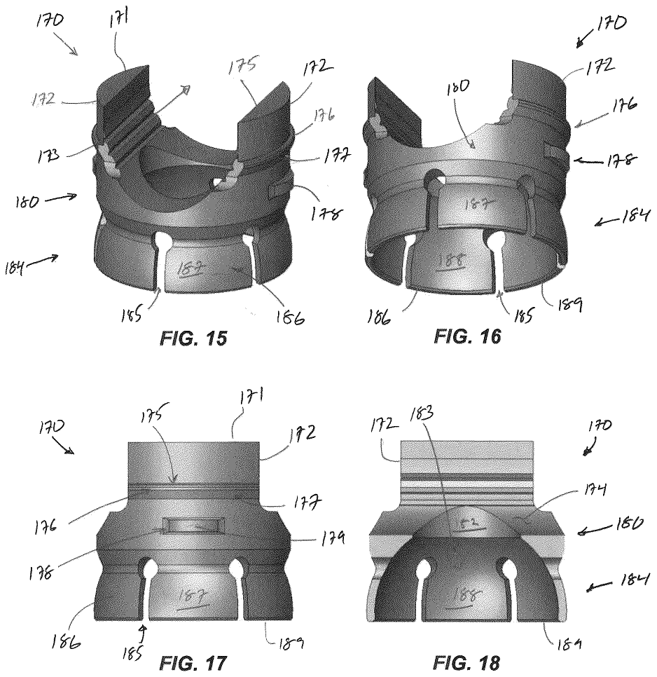

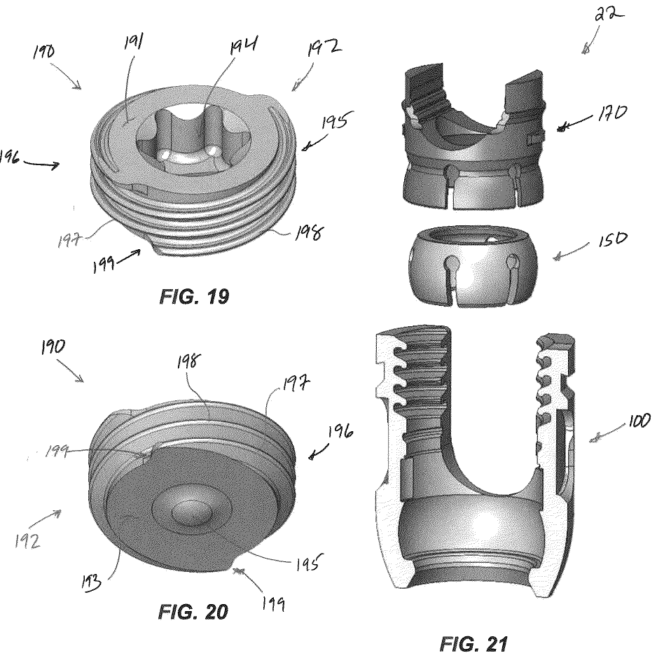

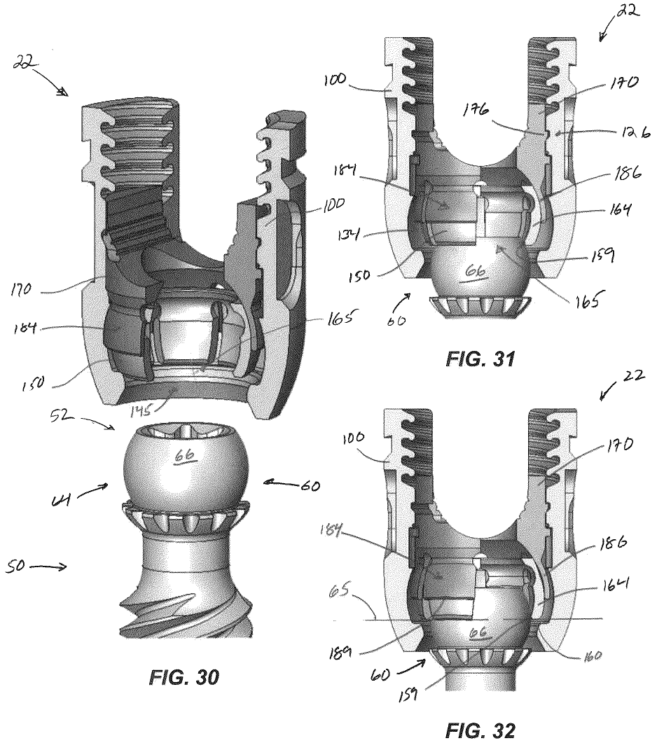

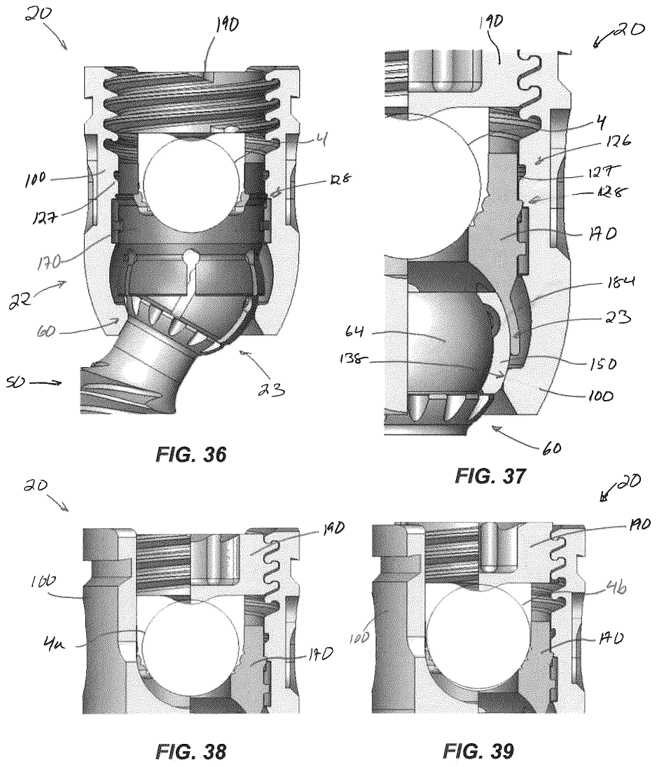

Preceding FIGS. 5-8 and following FIGS. 11-39 illustrate the different components of the multiplanar receiver sub-assembly 22 and their assembly together to form the fully-assembled multiplanar bone anchor assembly 20 shown in FIG. 1(b), 9-10 and 36-39. For instance, the cap retainer 150 and its connection with the bi-spheric shank head 60 are shown in FIGS. 5-8, the multiplanar receiver 100 or housing is shown in FIGS. 11-14, the single-piece, twist-into-position and downwardly displaceable collet insert is shown in FIGS. 15-18, and the single piece closure 190 is shown in FIG. 19-20. The pre-assembly of the cap retainer 150 and the collet insert 170 into the multiplanar receiver 100 to form the multiplanar receiver sub-assembly 22 in the shipping state configuration is shown in FIGS. 21-29. It is notable that the downwardly-extending collet fingers of the lower collet portion of the collet insert 170 are sized and shaped to extend below the hemisphere plane of the cap retainer 150, so as grip the cap retainer in a stabilizing and centralizing manner above the bottom opening of the receiver in the shipping state configuration. After the uploading of the bi-spheric shank head or capture structure 60 through the bottom opening and into the cap retainer, as shown in FIGS. 30-33, the collet insert, cap retainer and capture structure can be downwardly deployed with a tool or tooling (not shown) within the multiplanar receiver 100 to a non-floppy friction fit configuration, as shown in FIGS. 34-35. The capability of the upward-facing insert channel of the collet insert 170, the upper portion of the receiver 100, and the closure 190 to accommodate both a 5.5 mm and a 6.0 mm diameter rod in the fully assembled and locked configuration, is illustrated in FIGS. 36-39.

Returning now to FIGS. 5-6, the multiplanar cap retainer 150 can have the form of a hollow, partial spherical shell with a solid or continuous upper ring portion 154 having an annular planar upper surface 152 with a continuous circular inner edge 151 that defines a central upper opening 155. As can be seen in the drawings, a discontinuous outer spherical surface 156 extends downward from the continuous circular outer edge 153 of the upper surface 152 toward a discontinuous annular bottom surface 160, and a discontinuous inner spherical surface 158 extends downward from the circular inner edge 151 of the upper surface 152 toward the discontinuous annular bottom surface 160. The distance between the discontinuous inner spherical surface 158 and the discontinuous outer spherical surface 156 can define the thickness of the partial spherical shell that forms the cap retainer. A plurality of slots 162 can be formed through the thickness of the cap retainer 150, from the discontinuous inner spherical surface 158 to the discontinuous outer spherical surface 156 and extending upward from the discontinuous annular bottom surface 160 toward the continuous circular upper ring portion 154. The slots 162 can be equally spaced around the circumference of the cap retainer 150 to form a plurality of flexible collet fingers 164 extending downward from the upper ring portion, and which collet fingers 164 can flex outwardly at their lower ends, so as to expand a central lower opening 165 of the cap retainer 150 to receive the bi-spheric shank head of the shank. It will be appreciated that the discontinuous outer spherical surface 156, the discontinuous inner spherical surface 158, and the discontinuous annular bottom surface 160 can be considered ‘discontinuous’ due to the interruptions in the surfaces created by plurality of slots extending upwardly through the lower edge and thickness of the lower portions of the shell forming the cap retainer 150, and that other terminology may also be applicable.

As shown in the drawings, the cap retainer 150 can also include an inner beveled edge surfaces 159 between the discontinuous bottom annular surface 160 and the discontinuous inner spherical surface 158, which inner beveled edge surfaces 159 can define the expandable central lower opening 165 of the cap retainer. An outer beveled edge surface 161 can also be formed between the discontinuous bottom annular surface 160 and the discontinuous outer spherical surface 156. In addition, a rounded or curvate inner groove 166 can be formed into an upper portion of the discontinuous inner spherical surface 158 below the circular upper ring portion 154, which groove 166 can serve to reduce the thickness of cap retainer 150 near the roots of the downwardly-extending collet fingers 164 and thereby reduce stress in the material and facilitate the expansion of the collet fingers 164 during uploading of the bi-spheric shank head. In one aspect the upper ends of the slots 162 formed through the thickness of the cap retainer can also be formed as rounded apertures 163 or curved stress-relieving end passages.

The diameter of the discontinuous inner spherical surface 158 of the cap retainer 150 is substantially equal to the minor diameter 67 defined by the upper spherical surface 66 of the bi-spheric shank head 60 (see FIG. 4), while the diameter of the discontinuous outer spherical surface 156 is substantially equal to the major diameter 77 defined by both the lower spherical surface 76 of the bi-spheric shank head 60 and the spherical seating surface 136 of the receiver 100 (see FIGS. 11-14). As such, the cap retainer 150 can be sized and shaped so that once positioned on the bi-spheric shank head 60, as shown in FIGS. 7-8, the discontinuous inner spherical surface 158 can mate or engage with the upper spherical surface 66 while the discontinuous annular bottom surface 160 engages with the lower upward-facing shelf or ledge 72 of the bi-spheric shank head. In this coupled or captured configuration, in one aspect the annular planar upper surface 152 of the cap retainer 150 can be substantially flush or aligned with the annular planar top surface 62 of the bi-spheric shank head 60. In addition, the discontinuous outer spherical surface 156 of the cap retainer 150 can also be aligned with the lower spherical surface 76 of the lower partial spherical portion 74 so as to create a single diameter, articulating, multiplanar shank head sub-assembly 23 having the major diameter 77 that is greater than the diameter of the bottom opening 145 of the receiver 100, thereby preventing the bottom-loaded shank 50 from exiting the receiver 100 back out through the same bottom opening 145 through which it was initially loaded. It will be appreciated that the cap retainer 150 can still remain a member of the receiver sub-assembly 22 even after its coupling to the bi-spheric shank head 60 to form the shank head sub-assembly 23, and as such may be considered the linking mechanism that connects the two sub-assemblies together.

Illustrated in FIGS. 11-14 is the multiplanar receiver 100 having a generally U-shaped appearance with a partially discontinuous substantially cylindrical inner profile and a partially cylindrical and partially faceted outer profile, although other profiles are contemplated. For example, it is foreseen that this type of receiver can also be configured with planar lateral side surfaces. The receiver 100 generally comprises the base portion 140 defining the internal cavity 134 or lower portion of a generally cylindrical central bore 120 that is centered around the receiver's vertical centerline axis 101, and the pair of upright arms 110 extending upwardly from the base 140 to form the upper portion of the receiver and to define the upwardly-open rod channel 106 that is configured for receiving the elongate rod. Each of the upright arms 110 has an interior face 104 that includes a discontinuous upper portion of the central bore 120, which may be bounded on either side by opposed vertically-aligned planar end surfaces 107 that curve downwardly into U-shaped lower saddle surfaces 108. In one aspect the opposed planar end surfaces 107 and the curved saddle surfaces 108 can together define the front and back ends of the upwardly-open rod channel 106 that also opens laterally onto a front face 116 and a back face 117 of the receiver 100, respectively. From top surfaces 102 of the upright arms 110 at the proximal end 103 of the receiver, the central bore 120 can extend downwardly through both the rod channel 106 and the internal cavity 134 to communicate with a bottom surface 148 of the receiver through a bottom opening 145 at the distal end 147 of the receiver.

The upper or channel portion of the central bore 120 further includes a discontinuous guide and advancement structure 122 formed into the interior faces 104 of the upright arms 110, which guide and advancement structure 122 is configured to engage with a complementary structure formed into the outer side surfaces of the closure 190 (see FIGS. 19-20), as described more fully below. The guide and advancement structure 122 in the illustrated embodiment can be a discontinuous helically wound interlocking flange form 123. It will be understood, however, that the guide and advancement structure 122 could alternatively comprise a square-shaped thread, a buttress thread, a modified buttress thread, a reverse angle thread, or other thread-like or non-thread-like closure mating structure for operably guiding the closure downward between the upright arms 110 under rotation until the closure directly engages and presses against the elongate rod positioned within the channel 106. Additionally, the various structures and surfaces forming a helically wound guide and advancement structure 122 can also be configured to resist, to inhibit, to limit, or to preferentially allow and control some limited amount of splay of the upright arms 110 of the receiver 100 while advancing the closure downward under rotation and when torquing the closure against the elongate rod to generate a downwardly-directed thrust that locks the completely assembled multiplanar bone anchor assembly into position (see FIG. 9).

Moving downward along the interior faces 104 of the upright arms 110, the upper portion of the central bore 120 located between the vertical end surfaces 107 that define the channel 106 can include an upper discontinuous cylindrical surface 124 having an inner diameter that, in one aspect, can be substantially equal to the crest diameter of the helically-wound interlocking flange form 123. Alternatively, it is foreseen that the inner diameter of the upper discontinuous cylindrical surface 124 may be greater than or less than the crest diameter of the flange form 123, and that a run-out groove or grooves may also be formed into the interior faces 104 of the upright arms between the guide and advancement structure 122 and the upper discontinuous cylindrical surface 124.

With continued reference to FIGS. 11-14, the upper discontinuous cylindrical surface 124 be bisected into upper and lower portions by opposed shipping state grooves 126. The shipping state grooves 126 can extend across the width of the interior faces 104 of each upright arm 110 to the vertical end surfaces 107 that define the front and back ends of the channel 106. As described below, the shipping state grooves 126 can allow for opposite upper outer ridges of the collet insert 170 to be rotated into position within the shipping state grooves 126 from either direction. It is foreseen that other configurations for the opposed shipping state grooves are possible, including shipping state grooves with a center portion that only extends in one rotational direction (i.e., clockwise or counter-clockwise) to one of the vertical end surfaces 107, such that the opposite upper outer ridges of the collet insert 170 can only be rotated into position from that direction.

The upper surfaces 125 of the opposed shipping state grooves 126 can be downward-facing arcuate planar surfaces configured to engage with planar upper surfaces of the opposite upper outer ridges, so as to prevent the collet insert 170 from moving upward within the central bore 120 after the opposite upper outer rims have been rotated into position within the shipping state grooves 126. In contrast, the lower surfaces of the shipping state grooves 126 can comprise ramped surfaces 127 that extend downwardly and inwardly toward the lower portion of upper discontinuous cylindrical surface 124 located just below the shipping state grooves 126.

Moving downward through the central bore 120, a lower discontinuous cylindrical surface 130 can be located below the lower portion of the upper discontinuous cylindrical surface 124, with opposed lower locking grooves 128 being formed into the interior surfaces 104 of the upright arms 110 at the junction between the two discontinuous cylindrical surfaces. The lower discontinuous cylindrical surface 130 can have an internal diameter that is greater than the internal diameter of the upper discontinuous cylindrical surface 124, such that the upper surfaces 129 of the lower locking grooves 128 can be downward-facing arcuate planar surfaces configured to engage with the same planar upper surfaces of the opposite upper outer ridges of the collet insert 150 after the collet insert 150 has been pushed downwardly across the axial width of the lower portion of the upper discontinuous cylindrical surface 124, as described in more below.

The lower discontinuous cylindrical surface 133 can extend downward from the lower locking grooves 128 to a lower circumferential edge 131, and can include opposed vertically-elongate side pockets 132 formed into center portions of the lower discontinuous cylindrical surface 130 below the opposed lower locking grooves 128. In one aspect each side pocket 132 can be defined by a vertically-aligned, inwardly-facing arcuate sidewall surface that can be bounded above and below by upper and lower planar surfaces, respectively. The arcuate sidewall surface can be sized and shaped to generally match the profile of opposite indexing structures or nubs protruding from side surfaces of the collet insert 170, as described in more detail below. As disclosed in the present embodiment of the multiplanar receiver sub-assembly 22 illustrated in the drawing figures, for example, both the indexing nubs and the side pockets can have an arc-shaped profile.

Each side pocket 132 can also include one or more horizontal access recesses 133 extending from an upper portion of the side pocket 132 to an upper portion of the curved saddle surfaces 108 extending between the upright arms 110, so as to provide access to the side pockets 132 for the indexing nubs of the collet insert 150. In one aspect the depth of the horizontal access recesses 133 relative to the lower discontinuous cylindrical surface 130 can vary, and in particular can become slightly reduced or shallower, ramped, or increasingly-inwardly-sloped as moving from the opposed vertical planar end surfaces 107 toward the side pockets 132. This slight reduction in depth can create a resistance to the movement of the indexing nubs through the access recesses, and correspondingly a resistance to the rotation of the collet insert 170 about the centerline axis 101 of the receiver, and which resistance can be released as soon as the indexing nubs pass completely through the horizontal access recesses 133 and into the vertical side pockets 132. It will be appreciated that the reduced depth of the horizontal access recess 133 as it merges with the side pocket 132 can also serve to inhibit the indexing nubs from accidentally or unintentionally re-entering the horizontal access recesses 133 from within the side pocket after the collet insert 170 has been rotated into its aligned position with the channel 106 of the receiver 100.

With continued reference to FIGS. 11-14, the internal cavity 134 of the base portion 140 of the receiver 100 generally includes an upper expansion portion and a lower seating surface portion located proximate the bottom opening 145. The upper expansion portion is generally defined by the lower circumferential edge 131 demarking the lower end of the lower discontinuous cylindrical surface 130, a curvate sidewall surface 135 extending downwardly and around toward an annular, upward-facing upper step surface 136 that, in turn, extends inwardly to the upper circumferential edge of the spherical seating surface 138. As shown in the figures, in one aspect the expansion portion of the internal cavity 134 can have a round or spherical shape as viewed from above, although other shapes are also contemplated (such as oblong, squared with rounded corners, and the like) and considered to fall within the scope of the present disclosure.

The lower portion of the internal cavity 134 includes the spherical seating surface 138 that is slidably mateable with the spherically-shaped discontinuous outer surface of the cap retainer 150 and, at high angles of articulation of the bone anchor with respect to the receiver, with the lower spherical surface of the bi-spheric shank head 60. As shown in the drawings, the spherical seating surface 138 can be a partially spherically-shaped seating surface that extends downwardly below the inner circumferential edge 137 of the upward-facing upper step surface 136 to a lower circumferential edge 139 with a lowermost cylindrical surface 144 that can define the bottom opening 145 of the receiver 100. A lowermost tapered surface 146 of the central bore 120 can extend downwardly and outward from the lowermost cylindrical surface 144 to the bottom surface 148 of the receiver to provide a tapered approach to the bottom opening 145. It is foreseen that the other shapes or structures for the seating surface of the central bore, which are also slidably mateable with the multiplanar cap retainer that is configured to capture and hold the bi-spheric shank head or capture portion 60 of the bone anchor, are also possible, including but not limited to a non-spherical surface, a conical or tapered surface, a chamfered surface, a sharp edged or stepped lower structure, a cylindrical surface, and the like, and are considered to fall within the scope of the present disclosure. It is further foreseen that the seating surface could also be in a separate lower portion or part of the receiver that is subsequently attached to an upper part of the receiver.

The multiplanar receiver 100 can have a partially cylindrical and partially faceted outer profile. In the illustrated embodiment, for example, the partially cylindrical portions can include curvate side outer surfaces 112 of the upright arms 110 opposite the interior faces 104 that extend downward from the top surfaces 102 of the upright arms toward a lower outer tapered or curvate surface 141 of the base 140 that curves inwardly to the bottom surface 148 of the receiver 100. The receiver 100 can further include upper curvate-extending instrument engaging grooves 114 below the top surfaces 102 of the upright arms 110 that extend horizontally across the curvate side outer surfaces 112, and in one aspect (not shown) can extend to the front face 116 and the back face 117 of the receiver 100.

Likewise shown in the drawings, the faceted or planar portions of the receiver 100 may comprise front and back outer planar faces 142 on the receiver base 140 below the rod channel 106, and which can extend upwardly as narrow flats 418 or tool engagement features on the front and back faces 116, 117 of the upright arms 110. The faceted or planar portions of the multiplanar receiver 100 can further include side outer planar faces (not shown) and/or tool receiving and engaging side recesses 113 formed into the curvate side outer surfaces 112 below the upper instrument engaging grooves 114, and which can be parallel with each other and oriented perpendicular to the front and back outer planar faces 142. In one aspect the upper instrument engaging grooves 114, the narrow flats 118, the side recesses 413, the front and back outer planar faces 142, and any other planar tool-engagement surface or recess can serve together as outer tool engagement surfaces that allow for tooling to more securely engage and hold the receiver 100 during an initial pre-assembly with the internal components to form the multiplanar receiver sub-assembly 22, during coupling of the receiver sub-assembly to the bone anchor 50, either after or before the implantation of the anchor portion 84 of the bone anchor into a vertebra, and also during further assembly of the multiplanar receiver sub-assembly 20 with the elongate rod and the closure so as to aid in torquing and counter-torquing to lock the assembly.

Furthermore, it will be appreciated that the receiver 100 can also include additional features and aspects not shown in the drawings, including but not limited to inwardly-threaded breakoff extensions extending upwardly from the tops of the upright arms for interfacing with tooling and for guiding the elongate rod and the outwardly-threaded closure into the receiver channel. It is also foreseen that other shapes and configurations for the interior and exterior surfaces of the receiver 100, different from those shown in the drawings while providing for similar interaction and functionality of the various components of the pivotal bone anchor assembly, are also possible and considered to fall within the scope of the present disclosure, including but not limited to receivers having bottom openings with cut-out sections or slanted bottom surfaces that form oblique or expanded bottom openings, and the like, that provide for increased pivotal motion for the shank in at least one direction.

Illustrated in FIGS. 15-18 is the multiplanar collet insert 170 that generally includes a circular center portion 180, a pair of insert arms 172 extending upward from the circular center portion 180 to define an insert channel 173, and a lower collet portion 184 extending downward from the circular center portion 180 to define a discontinuous, downwardly-opening concave inner spherical surface 188 that is engageable with the discontinuous spherical outer surface of the cap retainer. The collet insert 170 can have a generally-cylindrical shape that is sized to be slidably received within the central bore of the multiplanar receiver. The insert arms 172 can form the insert channel 173 extending therebetween that is alignable with the rod channel of the receiver after the collet insert 170 has been positioned within the central bore, and which insert channel 173 can be further defined by an upward-facing rod seating surface 174 extending between the insert arms 172 that is engageable with the cylindrical elongate rod. The collet insert 170 can further include a central tool-receiving aperture 183 defined by an inner cylindrical surface 182 that is configured to slidably receive a drive tool (not shown) that extends downwardly through the central bore of the multiplanar receiver to engage the internal drive socket formed into the top end of the bi-spheric shank head.

The lower collet portion 184 can comprise a discontinuous curvate skirt extending downwardly and outwardly from the circular center portion 180 toward a discontinuous bottom edge surface 189, and having plurality of slots 185 formed through the thickness of the curvate skirt, from the discontinuous outer spherical surface 187 to the discontinuous inner spherical surface 188 and extending upward from the discontinuous bottom edge surface 189 toward the continuous circular center portion 180. The slots 185 can be equally spaced around the circumference of the curvate skirt to form a plurality of flexible collet fingers 186 extending downward from the circular center portion 180, and which collet fingers 186 can flex outwardly at their lower ends, so as to expand a central lower opening of the collet insert 170 to receive the cap retainer 150. It will be appreciated that the discontinuous outer spherical surface 187, the discontinuous inner spherical surface 188, and the discontinuous bottom edge surface 189 can be considered ‘discontinuous’ due to the interruptions in the surfaces created by plurality of slots 185 extending upwardly through the lower edge 189 and extending laterally through the thickness of the curvate skirt forming the lower collet portion 184, and that other terminology may also be applicable.

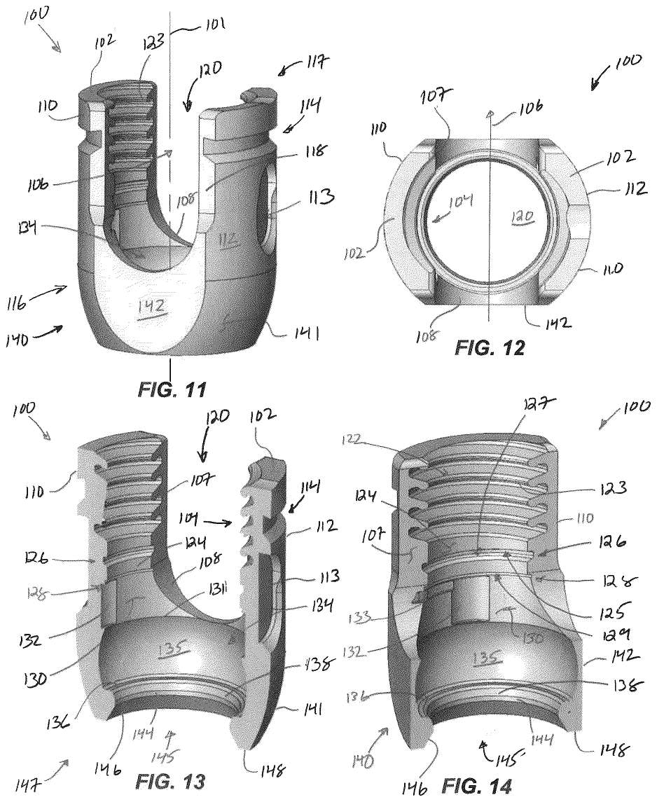

The insert arms 172 can include opposite upper outer ridges 176 that project radially outward from the outer surfaces of the insert arms 172 so as to rotatably slide into the upper shipping state grooves 126 formed into the central bore 120 of the multiplanar receiver 100 (see FIGS. 13-14) when the collet insert 170 is rotated about the vertical centerline axis 101 of the receiver 100. The upper outer ridges 176 can include upward-facing arcuate planar upper surfaces 175 configured to abutingly engage the downward-facing arcuate planar surfaces 125 of the opposite shipping state grooves 126, as well as downward-facing arcuate ramped or tapered lower surfaces 177 configured to slidably engage the ramped lower surfaces 127 of the shipping state grooves 126.

The multiplanar collet insert 170 may also include an indexing structure configured to releasably engage with a complementary indexing structure formed into the central bore of the multiplanar receiver, upon rotation of the collet insert 170 about the receiver vertical centerline axis, so as to inhibit further rotation of the insert out of its rotated position. For example, in one embodiment the indexing structure of the insert can comprise opposite outwardly-projecting indexing nubs 188 or protuberances projecting radially outward from the sides of the collet insert 180 below the opposite upper outer ridges 176. The indexing nubs 188 slidably rotate through the access recesses 133 to become positioned with the opposed vertical side pockets 132 formed into the central bore 120 of the multiplanar receiver 100 upon rotation of the collet insert 170 into its rotated or shipping state position. It is foreseen that other structures can be used to hold the insert relative to the receiver, such as crimps, pegs, set screws or separate rings, to inhibit rotational movement and/or to control translational movement of the insert along the vertical axis of the receiver, and that the insert could be snapped in place, or otherwise positioned, within the receiver.

Illustrated in FIGS. 19-20 is the single-piece closure 190 having a generally cylindrical closure body 192 with a top surface 191, a bottom surface 193, and an outer continuous guide and advancement structure 195 formed into the outer side surface 196 of the closure body 192 that operably joins with the discontinuous guide and advancement structure formed into the upright arms of the receiver. As illustrated, the outer continuous guide and advancement structure 195 can be a dual flange/dual lead-in helically wound interlocking flange having first and second closure flange forms 197, 198 and corresponding first and second closure starts 199. In one aspect the closure flange forms 197, 198 can include a splay-resisting or splay-controlling flange profile for operably guiding under rotation and advancing the closure 190 downward between the upright arms and having such a nature so as to resist, inhibit, limit, or preferentially control the splaying of the upright arms when the closure is advanced into the rod channel. In other embodiments not shown, the outer continuous guide and advancement structure can be a single flange/single lead-in guide and advancement structure having a single helically wound interlocking closure flange form and a corresponding single start, or may have more than two closure flange forms and corresponding starts. In other aspects, the guide and advancement structure may take on a variety of alternative forms, including but not limited to single closure thread/single start, dual closure threads/dual starts, buttress threads, square threads, reverse angle threads, interlocking gripping or dovetail-like threads, or other thread-like or non-thread-like helically wound advancement structures.

As shown in the drawings, in one aspect the bottom surface 193 of the closure 190 can include a downwardly-projecting central point 195 for engaging and securing the elongate rod. In other embodiments the bottom surface can include an annular projection, a point ring (i.e., an annular ring surrounding a central point or projection), a downwardly-projecting stepped planar surface for controlling the closure torque to thrust ratio, a recessed surface surrounded by a low outer ridge, and the like. In yet other embodiments the bottom surface can be substantially planar across the extent thereof. In yet other embodiments the closure can have a through-and-through central opening.

The top surface 191 of the closure 190 can further include a driving tool engagement structure, such as a central internal drive socket 194, which extends downward or inward into the body of the closure. The internal drive socket 194 can be used for closure installation or removal. Similar to the internal drive feature formed into the shank head, the internal drive socket of the illustrated closure is an aperture formed in the top surface, and in one aspect can be a multi-lobular or star-shaped aperture, such as those sold under the trademark TORX, or the like, having internal faces designed to receive a multi-lobular or star-shaped tool for rotating and driving the closure. It is foreseen that such a driving tool engagement structure may take a variety of tool-engaging forms and may include one or more apertures of various shapes, such as a plurality of bores of different diameters, a pair of spaced apart apertures or a hex shape designed to receive a hex tool (not shown) of an Allen wrench type. In one aspect the seat or base surface of the internal drive socket can be disposed perpendicular to a closure axis, with the internal drive socket otherwise being coaxial with the axis. In yet other embodiments the internal drive socket can extend entirely through the closure.

Illustrated in FIG. 21 are the individual components of the multiplanar bone anchor assembly that, in many embodiments, can be pre-assembled together into a multiplanar receiver sub-assembly 22 at a factory or manufacturing facility, prior to shipping to a spine company or a hospital or surgery center and engagement with the bi-spheric shank head of the bone anchor in the surgical setting. As described above, these components generally include the multiplanar receiver 100, the multiplanar cap retainer 150, and the multiplanar collet insert 170. In one aspect the multiplanar versions of the receiver 100, the cap retainer 150, and the collet insert 170 being pre-assembled into a multiplanar receiver sub-assembly 22 can be further defined as the shipping state configuration for the ‘modular’ multiplanar bone anchor assembly 20, as described herein and commonly understood in the art. It will be appreciated, however, that in other embodiments the shipping state configuration can include the additional assembly of the multi-planar receiver sub-assembly together with the bone anchor or shank at the factory or manufacturing facility or the spine company. It will also be appreciated that in yet other embodiments the individual components described above can also be pre-assembled into the receiver sub-assembly at the hospital or surgery center prior to implantation in a patient.

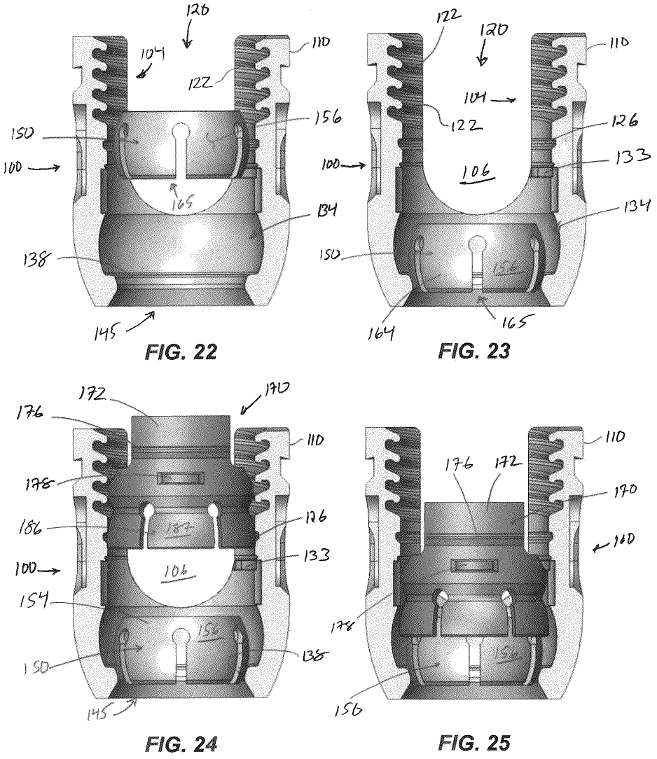

To begin the pre-assembly of the receiver sub-assembly, the cap retainer 150 can first be top-loaded into the receiver 100, as shown in FIGS. 22-23. With the cap retainer 150 having a major diameter that is less than the diameter of the outer surfaces 187 of the collet fingers 186 of the collet insert 170 and the upper portion of the central bore 120 of the receiver 100, and in particular less than the inner diameter of the guide and advancement structure 122 formed into the interior faces 104 of the upright arms 110 of the receiver 100, the cap retainer 150 can simply be downloaded through the central bore 120 in any orientation until the discontinuous outer spherical surface 156 engages with the spherical seating surface 138 at the lower end of the cavity 134 of the receiver 100. If needed, the cap retainer 150 can then be rotated to a horizontal position resting on the spherical seating surface 138, as shown in FIG. 23, with the central lower opening 165 defined by the inner beveled edge surfaces of the collet fingers 164 being centered adjacent to and aligned with the bottom opening 145 of the receiver 100.

After the cap retainer 150 is seated on the spherical seating surface 138 of the receiver 100, the collet insert 170 may then be top-loaded or down-loaded into the central bore 120 and installed into its the shipping state position above the cap retainer 150. As shown in FIGS. 24-25, this can be achieved by positioning the collet insert 170 above the central bore 120 with the insert arms 172 being aligned with the rod channel 106, and then downloading the collet insert 170 through the channel 106 (FIG. 24) until the opposite upper outer ridges 176 reach the level of the opposed shipping state grooves 126 (formed into the rod channel portion of the central bore 120 for this type of twist-in-place deployable collet insert 170) and the outwardly-projecting indexing nubs 178 reach the level of the horizontal access recesses 133. In this initial pre-rotation position the downwardly-opening concave inner spherical surface of the collet insert 170 is still spaced above the upper ring portion 154 and discontinuous outer spherical surface 156 of the cap retainer 150.

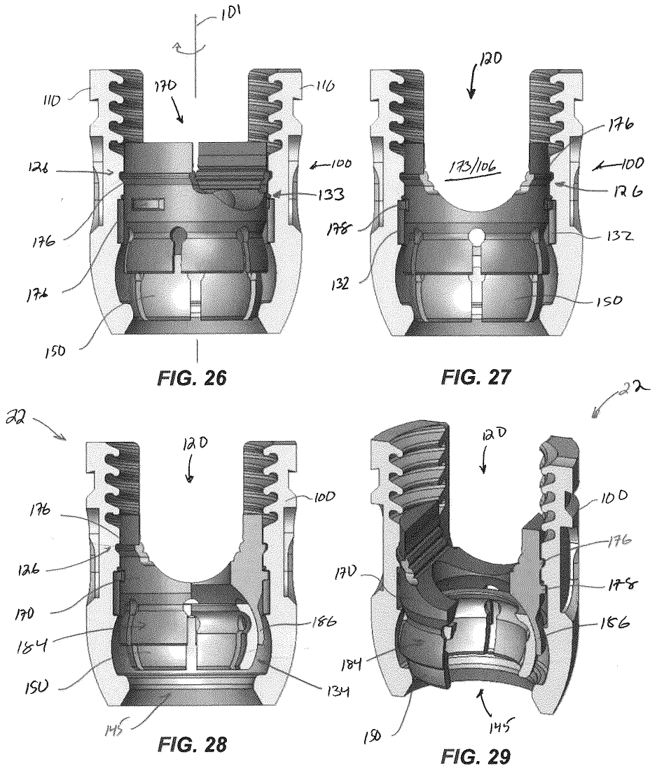

After reaching the initial downloaded position shown in FIG. 25, the collet insert 170 may then be rotated around its longitudinal axis (which is co-axial with the vertical centerline axis 101 of the receiver 100) so that the leading edges of the opposite upper outer ridges 176 begin to enter into the upper shipping state grooves 126 of the upright arms 110 and the outwardly-projecting indexing nubs 178 enter the horizontal access recesses 133, as shown in FIG. 26. The rotation of the collet insert 170 can continue for a full 90 degrees or quarter turn, until the insert channel 173 becomes aligned with the rod channel 106 of the receiver 100, the upper outer ridges 176 become fully centered within the upper shipping state grooves 126, and the indexing nubs 178 completely slide into the opposed vertical side pockets 132 of the central bore 120, as shown in FIG. 27. In one aspect the slightly reduced or shallower depth of the access recesses adjacent the vertical side pockets 132, as described above, can create some resistance to the rotation of the collet insert 180 toward the end of its rotation, so that the releasing of the indexing nubs 178 as they pass completely through the horizontal access recesses 133 and enter the vertical side pockets 132 can result in a ‘snap-in’ action which confirms that the insert sub-assembly 200 is properly positioned within the receiver 100.

With reference to FIGS. 28-29, and with the collet insert 170 securely positioned in the central bore 120 of the receiver 100 due to the upper outer ridges 176 being fully centered within the upper shipping state grooves 126, the cap retainer 150 can then be uploaded through the lower opening of the lower collet portion 184 defined by the discontinuous annular bottom surface 189, with the curvate collet fingers 186 flexing open within the expansion portion of the internal cavity 134 of the receiver 100, until the cap retainer 150 is captured by the lower collet portion 184 in a stabilized position that is suspended and centered over the bottom opening 145. The full rotation of the collet insert 170 into its initial position within the receiver 100 and the uploading of the cap retainer 150 into the lower collet portion 184, as shown in the drawings, can comprise the final steps for pre-assembling the multiplanar receiver sub-assembly 22 of the multiplanar bone anchor assembly. In addition, it will be appreciated that the receiver sub-assembly 22 is in its shipping state position or configuration that is configured to prevent both the collet insert 170 and the cap retainer 150 from exiting the central bore 120 of the receiver 100 and/or from moving out of alignment. In other words, the pre-assembly together of the multiplanar versions of the receiver 100, the cap retainer 150, and the collet insert 180 to form the multiplanar receiver sub-assembly 22 in the shipping state configuration, in which collet insert 180 is secured in an aligned position within the rod channel 106 and the cap retainer 150 is stabilized and centralized above the bottom opening 145, is now complete.

In one aspect the pre-assembly of the separate components into the multiplanar receiver sub-assembly 22, generally completed at the factory or manufacturing facility of the spine company, can be defined as the shipping state configuration of the ‘modular’ receiver sub-assembly 22, as described herein and commonly understood in the art. For example, in this configuration the multiplanar receiver sub-assembly 22 is now ready for storage and/or shipping and handling, and for eventual attachment to the bi-spheric shank head 60 of a bone anchor or shank 50 either prior to or during spinal surgery. Nevertheless, it will also be appreciated that in other embodiments the shipping state configuration can include the additional assembly of the multi-planar receiver sub-assembly 22 together with the bone anchor or shank 50 at the factory or manufacturing facility, with the pre-assembled multiplanar bone anchor assembly 20 then being shipped in trays, generally together with the closures 190, to the hospital or surgery center. It will also be appreciated that in yet other embodiments the individual components described above can also be pre-assembled into the receiver sub-assembly 22 and/or bone anchor assembly 20 at the hospital or surgery center prior to implantation in a patient.

It is foreseen that other structures for holding the collet insert 170 in alignment with the central bore 120 of the receiver 100 are also possible and considered to fall within the scope of the present disclosure, including but not limited to a reversal of the male/female relationship with inwardly-protruding projections being formed on an inner surface of the central bore and recesses or notches being formed into the outer surface of the collet insert.

One representative embodiment or method of assembling the multiplanar receiver sub-assembly 22 to the bi-spheric shank head 60 of the bone anchor or shank 50 is illustrated in FIGS. 30-35. For instance, and with initial reference to FIG. 30, the receiver sub-assembly 22 can be first positioned above the proximal end 52 of the bone anchor 50 with the expandable central lower opening 165 of the cap retainer 150, which is stabilized and centered above the bottom opening 145 of the receiver 100 by the lower collet portion 184 of the collet insert 170, being generally aligned with the upper partial spherical portion 64 of the bi-spheric shank head 60. The receiver sub-assembly 22 is then dropped downward (or the bone anchor is moved upward, depending on the frame of reference of the reader) until the upper spherical surface 66 of the bi-spheric shank head 60 passes upward through the bottom opening 145 of the receiver 100 toward the central lower opening 165.

As shown in FIG. 31, the receiver sub-assembly 22 continues to move downward (or the bone anchor moves upward) as the bi-spheric shank head 60 begins to push against the beveled edge surfaces 159 of the collet fingers 164 of the cap retainer 150 that is held in space within the internal cavity 134 by the lower collet portion 184 of the collet insert 170. The collet insert 170, in turn, is itself is upwardly immovable due to its engagement with the receiver 100 (via the upper outer ridges 176 being centered within the upper shipping state grooves 126). Due to this series of direct rigid engagements, the cap retainer 150 does not move upward and instead the central lower opening 165 of the cap retainer 150 can expand as both the collet fingers 164 of the cap retainer 150 and the collet fingers 186 of the collet insert 170 are pushed apart by the upwardly-moving bi-spheric shank head 60. At the same time, the inner beveled edge surfaces 159 of the collet fingers 164 can scrape downwards across the upper spherical surface 66 of the bi-spheric shank head 60, pushing any bone debris and/or soft tissue located on the outer surface downwards before them.

The collet fingers 164 of the cap retainer 150 and the collet fingers 186 of the collet insert 170 continue to be expanded within the upper expansion portion of the internal cavity 134 by the upward movement of the bi-spheric shank head 60 into the receiver 100. The expansion of the cap retainer 150 can continue, with the inner beveled edge surfaces 159 and/or the discontinuous annular bottom surface 160 of the cap retainer 150 pushing any bone debris and/or soft tissue located on the upper spherical surface 66 downwards before it, until the discontinuous annular bottom surface 160 of the cap retainer 150 reaches the level of the hemisphere plane 65 of the bi-spheric shank head 60 and the collet fingers 164 of the cap retainer 150 are at their point of maximum expansion, as shown in FIG. 32.

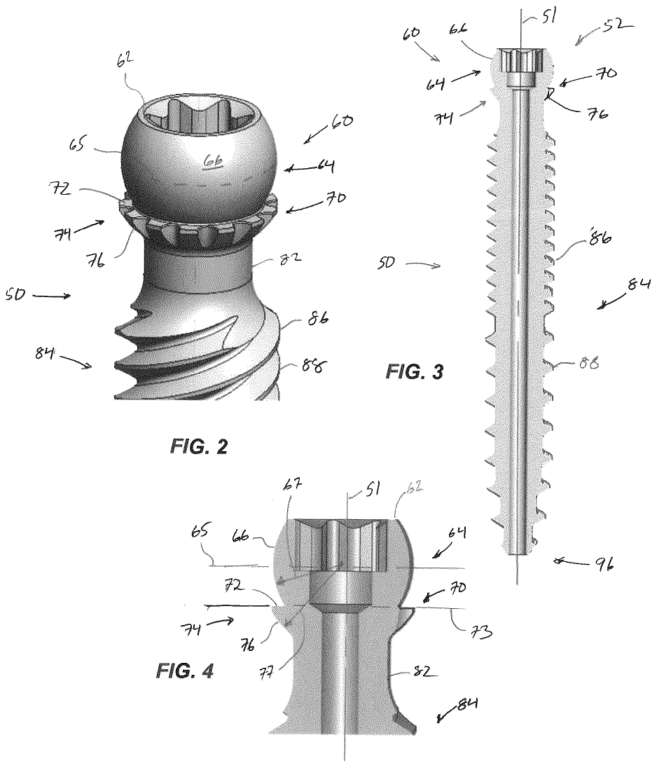

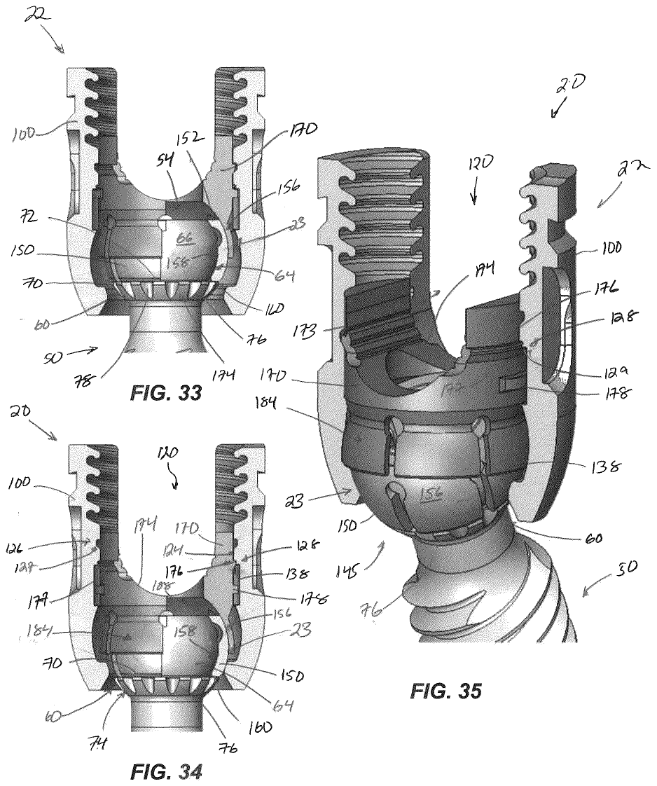

With reference to FIG. 33, the receiver sub-assembly 22 continues to move downward (or the bone anchor moves upward) until the upper partial spherical portion 64 of the bi-spheric shank head 60 becomes fully captured by the cap retainer 150 as the cap retainer 150 contracts to close around the upper spherical surface 66. During this motion, the discontinuous annular bottom surface 160 continues to push any bone debris and/or soft tissue downward toward the annular lower ledge 70 as the lower partial spherical portion 74 now moves upward into and through the bottom opening 145 of the receiver 100. Any bone debris and/or soft tissue that has been removed from the upper spherical surface 66 of the bi-spheric shank head 60 can pass through the plurality of open, vertically aligned flutes 78 that extend downwardly through and below the lower ledge 70 as the discontinuous annular bottom surface 160 engages the upward-facing planar surface 72 of the lower ledge 70 (see also FIGS. 2-4).

With continued reference to FIG. 33, the discontinuous inner spherical surface 158 of the cap retainer 150 is now secured around the upper partial spherical portion 64 of the bi-spheric shank head 60. Furthermore, with the simultaneous engagement of the discontinuous bottom annular surface 160 against the lower ledge 70 of the lower partial spherical portion 74, the cap retainer 150 is also now aligned on the bi-spheric shank head 60 so that the annular planar upper surface 152 that defines the central upper opening of the cap retainer 150 can be centered about the internal drive feature or drive socket 54 of the bi-spheric shank head 60. In addition, the discontinuous outer spherical surface 156 of the cap retainer 150 can also be aligned with the lower spherical surface 76 of the lower partial spherical portion 74 so as to create the single diameter, articulating, multiplanar shank head sub-assembly 23 having the major diameter 77 (see FIG. 8) that is greater than the diameter of the bottom opening 145 of the receiver 100, thereby preventing the bottom loaded shank 50 from exiting the receiver 100 back out through the same bottom opening 145 through which it was initially loaded. It will be appreciated that the cap retainer 150 can still remain a member of the receiver sub-assembly 22 even after its coupling to the bi-spheric shank head 60 to form the shank head sub-assembly 23, and as such may be considered the linking mechanism that connects the two sub-assemblies together.

With the shank head sub-assembly 23 secured within the lower collet portion 184, the collet insert 170 can then be downwardly deployed with a deployment tool (not shown). In one aspect the deployment tool can include a rounded lower surface that is complementary with the upward-facing rod seating surface 174 of the insert channel 173 of the collet insert 170. However, it is foreseen that a variety of other structural features for providing contact engagement between the deployment tool and the collet insert 170 are also possible and considered to fall within the scope of the present disclosure.

With reference to FIGS. 34-35, the deployment tool can be used to drive the collet insert 170 downward within the central bore 120, which can push the ramped lower surfaces 177 of the upper outer ridges 176 downward along the ramped lower surfaces 127 of the shipping state grooves 126 until the upper outer ridges 176 reach and scrape across the lower portion of the upper cylindrical surface 124, after which the upper outer ridges 176 snap under the upper arcuate planar surfaces 129 of the lower locking recess 128. The vertical or axial length of the side pockets 138 of the central bore 120 can also accommodate the downward movement of the indexing nubs 178 of the collet insert 170 positioned therein while maintaining the alignment of the collet insert 170 relative to the receiver 100. With the same motion the lower collet portion 184 and the captured shank head sub-assembly 23 are also driven downward until the lower portion of the discontinuous outer spherical surface 156 of the cap retainer 150 becomes engaged within the spherical seating surface 138 of the receiver 100.

It will be appreciated that the timing of the engagements between the upper outer ridges 176 and the upper arcuate planar surfaces 129 of the lower locking recess 128, and between the discontinuous outer spherical surface 156 and the spherical seating surface 138, can be substantially simultaneous. Upon completion of the deployment and removal of the deployment tool, as shown in FIGS. 34-35, the upper and lower engagements can serve to secure the collet insert 170 and the cap retainer 150 to the internal structures of the central bore 120, and thereafter prevent these components of the receiver sub-assembly 22 from moving back up (or down) within the receiver 100. The coupling of the universal bi-spheric shank head 60 of the bone anchor or shank 50 with the multiplanar receiver sub-assembly 22 can complete the formation of the multiplanar bone anchor assembly 20 in its initial configuration, one in which the multiplanar bone anchor assembly 20 is ready to be implanted into the vertebrae of a patient or to receive the elongate rod and the closure.

FIGS. 34-35 provide partially-sectioned views of the multiplanar bone anchor assembly 20 upon the initial assembly of the multiplanar receiver sub-assembly 22 to the bi-spheric shank head 60, but prior to final assembly with the elongate rod and the closure top. In one aspect the articulating multiplanar shank head sub-assembly 23 (i.e. the multiplanar cap retainer 150 and the bi-spheric shank head 60) can be secured against the downwardly-opening concave inner spherical surface 188 of the collet insert 170 and the spherical seating surface 138 of the multiplanar receiver 100 with a non-floppy frictional engagement, or pre-lock friction fit, that allows for the bone anchor or shank 50 to both pivot and rotate relative to the receiver 100 as the outer surfaces of the shank head sub-assembly 23 (i.e., the discontinuous outer spherical surface 156 of the cap retainer 150 and the lower spherical surface 76 of the bi-spheric shank head 60) slidably frictionally engage, with some resistance, with the concave inner spherical surface 188 and the spherical seating surface 138 with a ball and socket-type connection. It will be appreciated that this friction fit can be provided by the engagement between the upper outer ridges 176 of the collet inset 180 and the upper arcuate planar surfaces 129 of the lower locking recess 128 which, in turn, can be configured to provide a downwardly directed force to upper portions of the discontinuous outer spherical surface 156 of the cap retainer 150. This can create the initial non-floppy frictional engagements between the cap retainer 150, the lower collet portion 184 of the collet insert 170, and the spherical seating surface 138 of the multiplanar receiver 100 that allows for movement of the shank head sub-assembly 23 relative to the receiver 100 with some resistance.

Illustrated in FIGS. 36-37 is the multiplanar bone anchor assembly 20 after final assembly with the elongate rod 4 and the single piece closure 190, and in FIGS. 38-39 with differently-sized elongate rods 4a, 4b being positioned within the insert channel 173 of the collet insert 170. In these configurations the collet insert 170 can be pressed further downward within the receiver 100 by the elongate rod 4 and closure 190 so as to increase the downwardly directed force applied to the shank head sub-assembly 23. In one aspect the lower locking recesses 128 can include ramped lower surfaces (not numbered) that are smaller than the ramped lower surfaces 127 of the upper shipping state grooves 126, thereby allowing the collet insert 170 to be easily pressed downward within the central bore until a hard or full frictional lock between shank head sub-assembly 23, the lower collet portion 184 of the collet insert 170, and the spherical seating surface 138 of the multiplanar receiver 100 is achieved, preventing further movement between the shank head sub-assembly 23 and the receiver 100. In addition, the rod seating surface 174 of the collet insert 170 can also be configured to accommodate either a 5.5 mm diameter rod 4a (FIG. 38) or a 6.0 mm diameter rod 4b (FIG. 39) in the fully assembled and locked configuration.

Finally, it will be appreciated that subsequent limited unthreading or backing-off of the single piece closure 190 from the receiver 100, without removing the elongate rod 4 or completely detaching the closure 190, can remove the additional downwardly directed force that was provided by the closure 190, thereby releasing the hard lock and re-establishing the non-floppy, friction fit configuration between the components of the multiplanar bone anchor assembly 20. A slight wiggling of the multiplanar receiver sub-assembly 22 can then serve to re-mobilize the multiplanar receiver sub-assembly 22 relative to the bi-spheric shank head 60 and allow its position to be adjusted prior to re-locking the multiplanar bone anchor assembly 20 in a new position with a hard lock using the closure 190.

### Independent Lock Bone Anchor Assembly

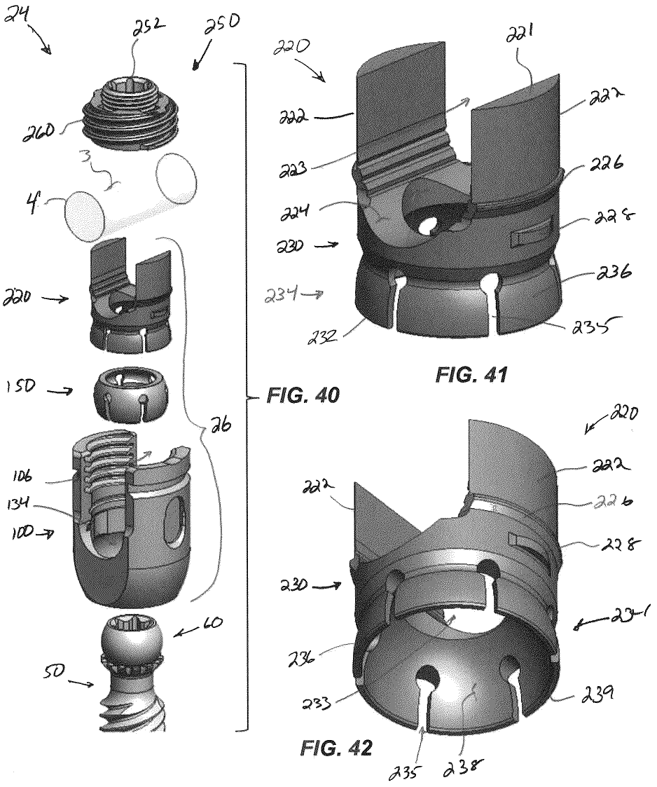

FIG. 40 is an exploded perspective view of the second representative embodiment 24 of the multiplanar bone anchor assembly illustrated in FIG. 1(c) that is configured to provide an independent lock (“IL”) functionality, in which the position of a multiplanar IL receiver sub-assembly 26 can be immovably locked to the bi-spheric shank head 60 of the bone anchor or shank 50 independent of the elongate rod 4 being locked within the channel of the multiplanar receiver 100. This multiplanar IL bone anchor assembly 24 can include the same bone anchor or shank 50 described above, having a bi-spheric shank head 60 and an anchor portion 84 opposite the bi-spheric shank head 60 for securement or attachment to the bone of a patient.

The multiplanar IL bone anchor assembly 24 can also include the same multiplanar receiver 100 that can be initially pivotably secured to the bi-spheric shank head 60 with a number of separate internal components that have been pre-assembled into the internal cavity 134 and the rod channel 106 of the receiver 100 to form the multiplanar IL receiver sub-assembly 26. These internal components can include, but are not limited to, the pivoting or articulating multiplanar cap retainer 150 and a multiplanar IL collet insert 220. Before or after the elongate rod 4 has been positioned within the lower portion of the rod channel 106, a two-piece closure 250 can be threadably or otherwise secured into an upper portion of the rod channel 106 to separately apply pressure to upper surfaces of the multiplanar IL collet insert 220 and to the upper surface 3 of the elongate rod 4, eventually locking both the elongate rod 4 and the multiplanar IL receiver sub-assembly 26 into a final locked position relative to the bone anchor or shank 50.

In other words, the primary difference between the multiplanar IL bone anchor assembly 24 and the previous embodiment can be the alternative IL collet insert 220 and the alternative two-piece closure 250 that function together with the receiver 100 to provide the independent lock functionality. It will be appreciated, moreover, that the multiplanar receiver 100 and multiplanar cap retainer 150 of the IL multiplanar bone anchor assembly 24 shown in FIG. 1(c), 40 and 64-66 can be the same as or substantially similar to those included in the multiplanar bone anchor assembly 20 described above, hence providing an additional degree of component-type modularity that can reduce the number of different individual components required to manufacture and assemble a spinal construct using the spinal fixation system 10 (see FIG. 1) of the present disclosure.

FIGS. 41-66 illustrate the different components of the multiplanar IL receiver sub-assembly 26 and their assembly together to form the multiplanar IL bone anchor assembly 24 shown in FIG. 1(c), 40 and 64-66. In particular, the single-piece, twist-into-position and downwardly displaceable multiplanar IL collet insert 220 is shown in FIGS. 41-44, and the two-piece closure 250 is shown in FIGS. 45-50.

With the exception that the insert arms 222 can extend further upward to define a top surface 221 that is configured to be engaged by the outer ring 260 of the two-piece closure 250, it is notable that the multiplanar IL collet insert 220 can have substantially the same construction and features of the multiplanar collet insert 170 described above. For example, and with reference to FIGS. 41-44, the multiplanar IL collet insert 220 can also include the circular center portion 230, the pair of insert arms 222 extending upward from the circular center portion 230 to define the insert channel 223, and the lower collet portion 234 extending downward from the circular center portion 230 to define a discontinuous, downwardly-opening concave inner spherical surface 238 that is engageable with the discontinuous spherical outer surface of the cap retainer. The circular center portion 230 can also include the central tool-receiving aperture 233 defined by an inner cylindrical surface 232, and the insert arms 222 can include the radially-outward projecting opposite upper outer ridges 226 and the indexing nubs 228 that are configured to rotatably slide into the upper shipping state grooves 126 and the lower accesses 132 formed into the central bore 120 of the multiplanar receiver 100, respectively, when the collet insert 220 is rotated about the vertical centerline axis 101 of the receiver 100 (see FIGS. 13-14). Finally, the lower collet portion 234 can also comprise the discontinuous curvate skirt extending downwardly and outwardly from the circular center portion 230 and having plurality of slots 235 equally spaced around the circumference of the curvate skirt to form a plurality of flexible collet fingers 236 extending downward from the circular center portion 230 toward the discontinuous bottom edge surface 239.

With reference to FIGS. 45-50, the two-piece closure 250 can include an outer ring 260 comprising a generally cylindrical body 262 having an annular top surface 261, an annular bottom surface 263, and a central through-aperture 264. A continuous guide and advancement structure 266 with a start structure 267 can be formed into the side surfaces of the cylindrical body 262 and configured to rotatably mate with the discontinuous guide and advancement structure formed into interior faces of the upright arms of the receiver. In one aspect, the guide and advancement structure 266 can be a helically-wound interlocking flange that is mateable with the complementary helically-wound interlocking flange formed into the multiplanar receiver 100. Nevertheless, and as described above with reference to the receiver, other versions of the continuous guide and advancement structure 266 complementary with that formed into the multiplanar receiver are also possible and considered to fall within the scope of the present disclosure.

The central through-aperture 264 of the outer ring 260 includes an internal guide and advancement structure, in this case an internal thread 268, that is configured to threadably receive the center screw 252. As can be seen in the drawing figures, the center screw 252 also comprises a generally cylindrical body 254, but one that is much smaller and having an external or outer thread 258 that is complementary with the internal thread 268 of the central through aperture 264. The center screw 252 further includes an annular top surface 253, a solid or continuous bottom surface 255, and a central closed-off aperture 256 formed as a drive structure or internal drive socket 257 extending downwardly from the annular top surface 253 toward the bottom surface 255. The outer ring 260 can also have a drive structure, in this case a plurality of downward-extending recesses 265 formed into the upper portion of the central through-aperture 264, and which may interrupt the upper portions of the internal thread 268.

As discussed in more detail below, the annular bottom surface 263 of the outer ring 260 is configured to engage with the top surfaces 221 of the insert arms 262 of the IL collet insert 220, while the closed-off bottom surface 255 of the center screw 252 is configured to engage the elongate rod. Other aspects of the two-piece closure 250 will be apparent to one of skill in the art upon further review of the drawing figures.

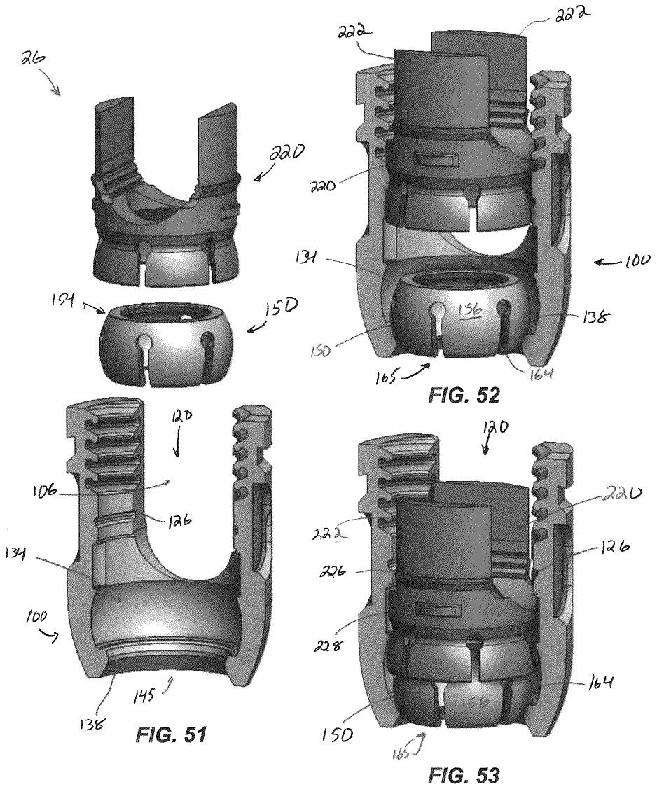

The pre-assembly of the cap retainer 150 and multiplanar IL collet insert 220 into the multiplanar receiver 100 to form the multiplanar IL receiver sub-assembly 26 in the shipping state configuration is shown in FIGS. 51-57, and can be substantially similar to the pre-assembly of the multiplanar receiver sub-assembly 22 described above. With initial reference to FIG. 52, for example, the cap retainer 150 can first be top-loaded into the receiver 100 until the discontinuous outer spherical surface 156 engages with the spherical seating surface 138 at the lower end of the cavity 134 of the receiver 100 and the central lower opening 165, defined by the inner beveled edge surfaces 159 of the collet fingers 164, is centered adjacent to and aligned with the bottom opening 145 of the receiver 100. After the cap retainer 150 is seated on the spherical seating surface 136 of the receiver 100, the IL collet insert 220 may then be top-loaded or down-loaded into the central bore 120 and installed into its the shipping state position above the cap retainer 150. This can be achieved by positioning the IL collet insert 220 above the central bore 120 with the insert arms 222 being aligned with the rod channel 106 (FIG. 52), and then downloading the IL collet insert 220 through the channel 106 until the opposite outer ridges 226 reach the level of the upper shipping state groove 126 and the outwardly-projecting indexing nubs 228 reach the level of the horizontal access recesses 132 (FIG. 53). In this initial pre-rotation position the concave lower surface 238 of the IL collet insert 220 is still spaced above the upper ring portion 154 and discontinuous outer spherical surface 156 of the cap retainer 150.

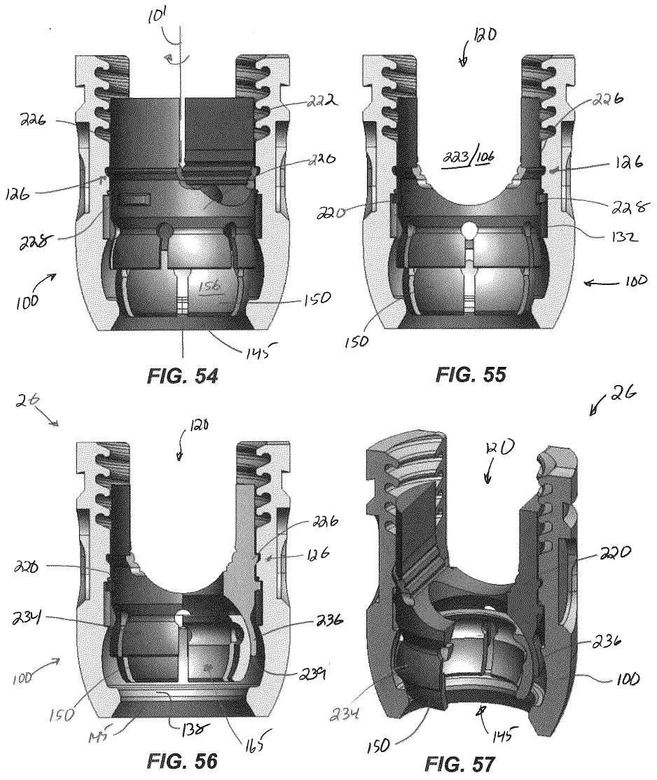

After reaching the initial downloaded position shown in FIG. 53, the IL collet insert 220 may then be rotated around its longitudinal axis (which is co-axial with the vertical centerline axis 101 of the receiver 100) so that the leading edges of the opposite upper outer ridges 226 begin to enter into the upper shipping state grooves 126 of the upright arms 110 and the outwardly-projecting indexing nubs 228 enter the horizontal access recesses 133, as shown in FIG. 54. The rotation of the IL collet insert 220 can continue for a full 90 degrees or quarter turn, until the insert channel 223 becomes aligned with the rod channel 106 of the receiver 100, the upper outer ridges 226 become fully centered within the upper shipping state grooves 126, and the indexing nubs 28 completely slide into the opposed vertical side pockets 132 of the central bore 120, as shown in FIG. 55.

With reference to FIGS. 56-57, and with the IL collet insert 220 securely positioned in the central bore 120 of the receiver 100 due to the upper outer ridges 226 being fully centered within the upper shipping state grooves 126, the cap retainer 150 can then be uploaded through the lower opening of the lower collet portion 234 defined by the discontinuous annular bottom surface 239. As described above, the upward movement of the cap retainer can cause the curvate collet fingers 236 to flex open within the expansion portion of the internal cavity 134 of the receiver 100, until the cap retainer 150 is captured by the lower collet portion 234 in a stabilized position that is suspended and centered over the bottom opening 145. The pre-assembly together of the multiplanar receiver 100, the multiplanar cap retainer 150, and the IL collet insert 220 to form the multiplanar IL receiver sub-assembly 26 in the shipping state configuration, in which the IL collet insert 220 is secured in an aligned position within the rod channel 106 and the cap retainer 150 is stabilized and centralized above the bottom opening 145, is now complete.

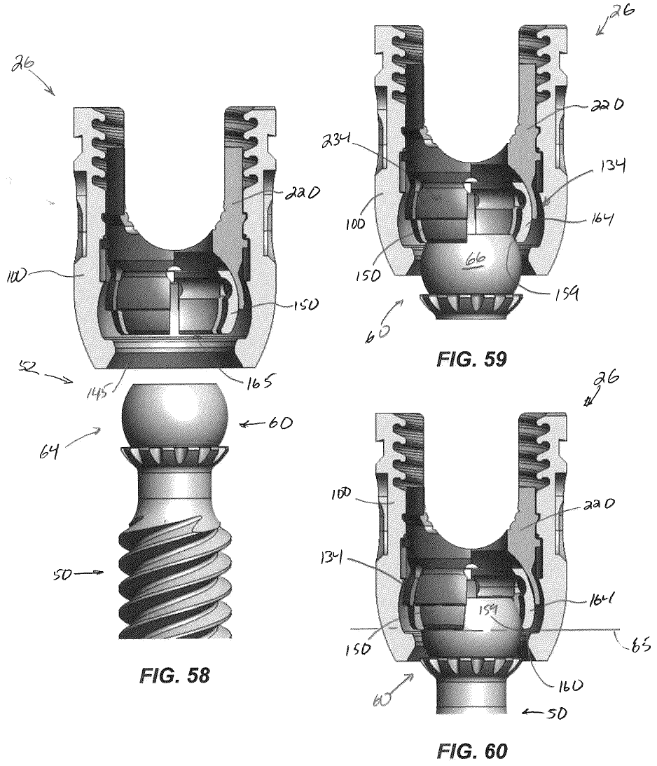

The assembly of the multiplanar IL receiver sub-assembly 26 with the bi-spheric shank head 60 of the bone anchor or shank 50 is illustrated in FIGS. 58-63, and can be substantially similar to the assembly of the multiplanar bone anchor assembly described above. With initial reference to FIG. 58, for example, the IL receiver sub-assembly 26 can be first positioned above the proximal end 52 of the bone anchor 50 with the expandable central lower opening 165 of the cap retainer 150 being generally aligned with the upper partial spherical portion 64 of the bi-spheric shank head 60. As shown in FIG. 59, the IL receiver sub-assembly 26 can then dropped downward (or the bone anchor moved upward, depending on the frame of reference of the reader) until the upper spherical surface 66 of the bi-spheric shank head 60 passes upward through the bottom opening 145 of the receiver 100 to engage the beveled edge surfaces 159 of the collet fingers 164 of the cap retainer 150 that is held in space within the internal cavity 134 by the lower collet portion 234 of the collet insert 220

As shown in FIG. 60, the IL receiver sub-assembly 26 continues to move downward (or the bone anchor moves upward) as the bi-spheric shank head 60 pushes against the beveled edge surfaces 159 to expand both the collet fingers 164 of the cap retainer 150 and the collet fingers 236 of the collet insert 220 within the upper expansion portion of the internal cavity 134 until the discontinuous annular bottom surface 160 of the cap retainer 150 reaches the level of the hemisphere plane 65 of the bi-spheric shank head 60 and the collet fingers 164 of the cap retainer 150 are at their point of maximum expansion.

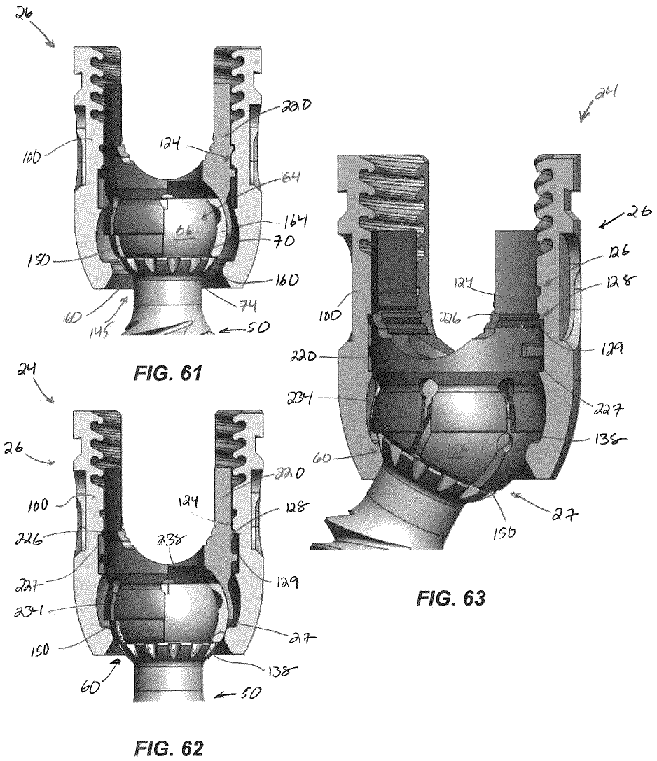

With reference to FIG. 61, the IL receiver sub-assembly 26 continues to move downward (or the bone anchor moves upward) until the upper partial spherical portion 64 of the bi-spheric shank head 60 becomes fully captured by the cap retainer 150. In particular, the collet fingers 164 of the cap retainer 150 can contract to close around the upper spherical surface 66 of the bi-spheric shank head 60 simultaneous with the discontinuous bottom annular surface 160 engaging the lower ledge 70 of the lower partial spherical portion 74 to create the single diameter, articulating, multiplanar IL shank head sub-assembly 27 having the major diameter 77 (see FIG. 8) that is greater than the diameter of the bottom opening 145 of the receiver 100.

After the uploading of the bi-spheric shank head or capture structure 60 through the bottom opening and into the cap retainer, the multiplanar IL collet insert 220, multiplanar cap retainer 150 and capture structure 60 can be downwardly deployed within the multiplanar receiver 100 with a tool or tooling (not shown), as shown in FIGS. 62-63. As with the first multiplanar embodiment described above, the deployment tool can be used to drive the IL collet insert 220 downward within the central bore 120, which can push the ramped lower surfaces 227 of the upper outer ridges 226 downward along the ramped lower surfaces 127 of the shipping state grooves 126 until the upper outer ridges 226 reach and scrape across the lower portion of the upper cylindrical surface 124, after which the upper outer ridges 226 snap under the upper arcuate planar surfaces 129 of the lower locking recess 128. With the same motion the lower collet portion 234 and the captured IL shank head sub-assembly 27 can also be driven downward until the lower portion of the discontinuous outer spherical surface 156 of the cap retainer 150 becomes engaged within the spherical seating surface 138 of the receiver 100, as shown in FIGS. 62-63. In one aspect the articulating multiplanar IL shank head sub-assembly 27 (i.e. the multiplanar cap retainer 150 and the bi-spheric shank head 60) can be secured against the downwardly-opening concave inner spherical surface 238 of the IL collet insert 220 and the spherical seating surface 138 of the multiplanar receiver 100 with a non-floppy frictional engagement, or pre-lock friction fit, as described above.

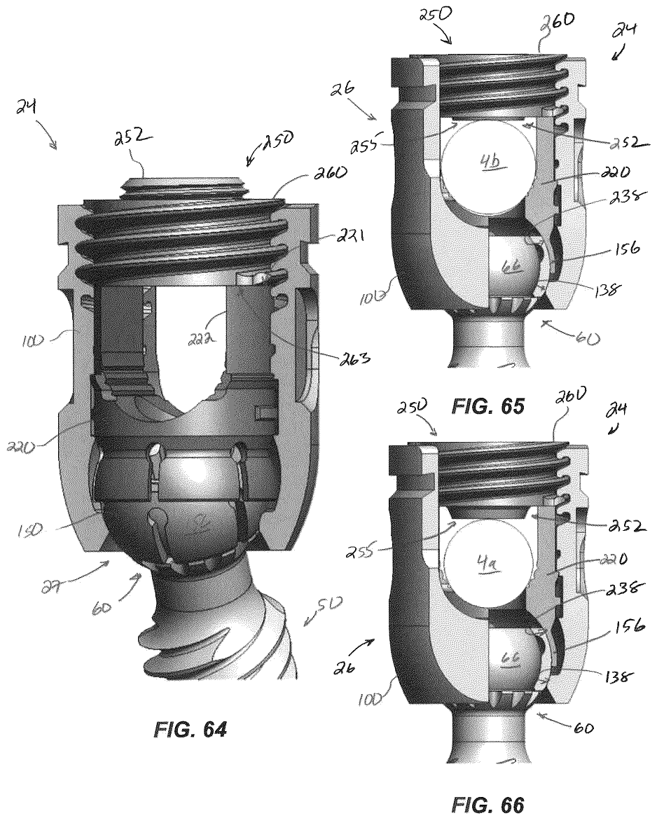

The independent lock capability provided by the IL collet insert 220 and the two-piece closure 250, while accommodating either a 5.5 mm rod 4a or a 6.0 mm diameter rod 4b in the fully assembled and locked configuration, is illustrated in FIGS. 64-66. For instance, as shown in FIG. 64, the annular bottom surface 263 of the outer ring 260 of the two-piece closure 250 (with or without the presence of the elongate rod) can engage the top surfaces 221 of the insert arms 222 of the IL collet insert 220, to drive the IL collet insert 220 further downward within the central bore 120. This downwardly-directed force can hard lock the outer spherical surface 156 of the cap retainer 150 between the lower concave inner surface 238 of the IL collet insert and the spherical seating surface 136 of the multiplanar receiver 100, so as to prevent further movement of the shank 50 relative to the IL receiver sub-assembly 28.

With reference to FIGS. 65-66, the inner set screw 152 of the two piece closure 250 can then be threaded downward within the outer ring 260 until the bottom surface 255 of the inner ring 252 engages the elongate rod 4a, 4b, to drive the elongate 4a, 4b rod downward into the insert channel 223 of the IL collet insert 280 and ultimately lock the elongate rod 4a, 4b within the multiplanar IL bone anchor assembly 24.

It will be appreciated that, similar to first multiplanar embodiment described above, subsequent limited unthreading or backing-off of the outer ring 260 of the two-piece closure 250 from the multiplanar receiver 100, without removing the elongate rod 4 or completely detaching the two-piece closure 250, can remove the additional downwardly directed force that was provided by the closure 250, thereby releasing the hard lock and re-establishing the non-floppy, friction fit configuration between the components of the multiplanar IL bone anchor assembly 24. A slight wiggling of the multiplanar IL receiver sub-assembly 26 can then serve to re-mobilize the multiplanar receiver sub-assembly 26 relative to the bi-spheric shank head 60 (and the cap retainer 150) and allow its position to be adjusted prior to re-locking the multiplanar IL bone anchor assembly 24 in a new position with a hard lock using the outer ring 260 of the two-piece closure 250.

### Monoplanar Bone Anchor Assembly

FIG. 67 is an exploded perspective view of one representative embodiment of the monoplanar bone anchor assembly 30 illustrated in FIG. 1(d) that is configured, as noted above, to limit the pivotal motion of the bone anchor 50 relative to the monoplanar receiver sub-assembly 32 (or vice versa) to a single plane while still providing for a 360-degree range of rotation around the longitudinal axis 51 of the bone anchor 50. The monoplanar bone anchor assembly 30 can include the same bone anchor or shank 50 described above, having the bi-spheric shank head 60 and the anchor portion opposite the bi-spheric shank head 60 for securement or attachment to the bone of a patient.

Similar to the multiplanar bone anchor assemblies discussed above, the monoplanar bone anchor assembly 30 can include a monoplanar receiver 300 that is initially pivotably secured to the bi-spheric shank head 60 with a number of separate internal components that have been pre-assembled into the internal cavity 334 and the rod channel 306 of the receiver 300 to form the monoplanar receiver sub-assembly 32. These internal components can include, but are not limited to, a pivoting or articulating monoplanar cap retainer 350 and a monoplanar collet insert 370. After the elongate rod 4 has been positioned within the lower portion of the rod channel of the monoplanar receiver 300, the same single-piece closure 190 (or another appropriate type of closure) can be threadably or otherwise secured into the upper portion of the rod channel to apply pressure to an upper surface of the elongate rod, thereby locking both the elongate rod and the monoplanar bone anchor assembly 30 into a final locked position.

Differences between monoplanar bone anchor assembly 30 of FIG. 67 and the multiplanar and multiplanar IL bone anchor assemblies 20, 24 described above can include the replacement of the multiplanar receiver with a monoplanar receiver 300, as shown in FIGS. 68-69. The monoplanar receiver 300 can have many of the same features as the multiplanar version, with the addition of an opposed pair of upward-facing pivot grooves 337 formed into the annular shelf surface 336 and the spherical seating surface 338 located in the lower portion of the internal cavity 334, and opposed expansion recesses 330 formed into the curvate sidewalls 335 located in the upper portion of the internal cavity 334. The monoplanar receiver 300 can also have enlarged opposed vertically-elongate side pockets 332 and enlarged access recesses 333 located below the downward-facing upper arcuate planar surfaces 329 of the lower locking grooves 328.

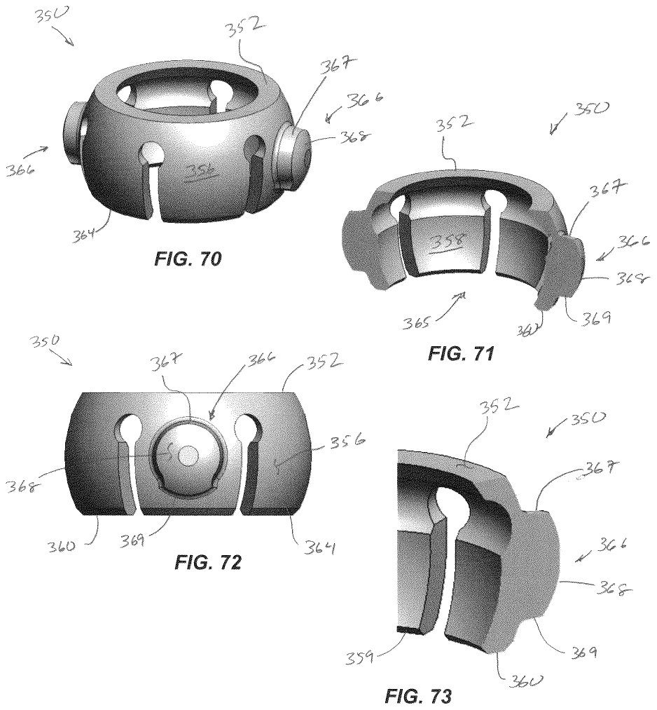

With reference to FIGS. 70-73, the additional differences can also include the replacement of the multiplanar cap retainer with a monoplanar cap retainer 350. The monoplanar cap retainer 350 can have many of the same features as the multiplanar version, with the addition of bi-circular opposite protrusions or pegs 366 that project outwardly from opposite sides of the discontinuous outer spherical surface 356. In one aspect the opposite pegs 366 can have rounded end surfaces 368, partial cylindrical upper surfaces 367, and lower curvate rocker surfaces 369, with the partial circular cylindrical upper surfaces 367 having a diameter that is less than the diameter of the lower curvate rocker surfaces 369. As describe in more detail below, the lower curvate rocker surfaces 369 and the rounded end surfaces 368 of the opposite pegs 366 are configured to be pivotably received within the opposed pivot grooves 337 of the monoplanar receiver 300, so that the pivotal motion of the monoplanar cap retainer 350 relative to the monoplanar receiver 300 is limited to a single plane defined by a pivot axis extending between the opposite pegs 366 positioned in the opposed pivot grooves 337.

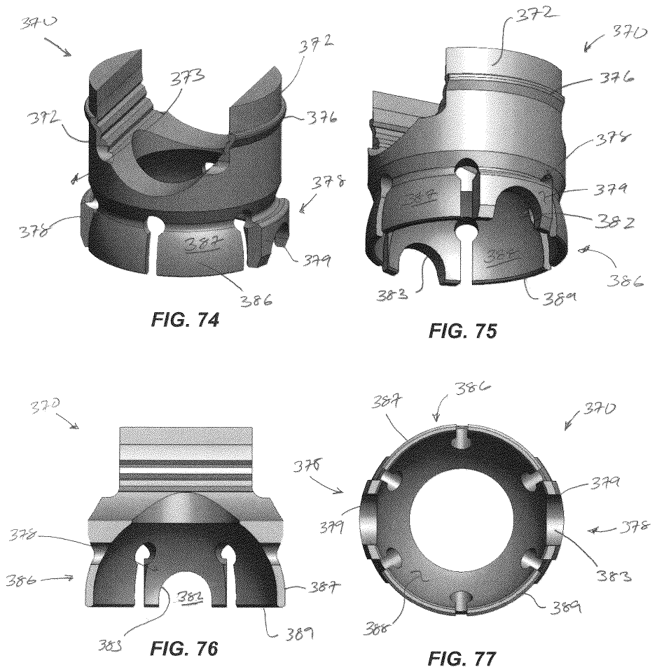

With reference to FIGS. 74-77, the additional differences can further include the replacement of the multiplanar versions of the collet insert with a monoplanar collet insert 370. The monoplanar collet insert 370 can have many of the same features as the multiplanar version, with the addition of opposite lower flange fingers 378 replacing the opposite collet fingers located directedly below the insert arms 372 of the monoplanar collet insert 370. The lower flange fingers 378 can include outer flange surfaces 379 having a slightly greater diameter than the maximum diameter of the outer collet surfaces 387 of the remaining collet fingers 386 of the lower collet portion 384. In one aspect the outer flange surfaces 379 of the lower flange fingers 378 can replace the function of the outwardly-projecting indexing nubs found in the multiplanar versions of the collet insert by sliding into the enlarged access recesses 333 and side pockets 332 to maintain the alignment of the monoplanar collet insert 370 relative to the monoplanar receiver 300. As can be seen in the drawing figures, the lower flange fingers 378 can also include circular cutouts 382 having a downward-facing partial cylindrical surfaces 383 configured to engage with the partial cylindrical upper surfaces 367 of the opposite pegs 366.

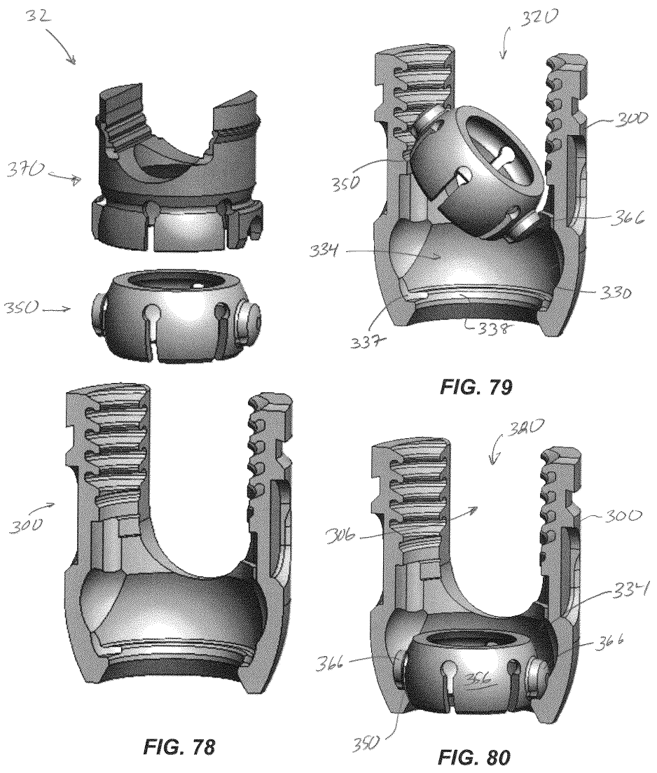

The pre-assembly of the monoplanar cap retainer 350 and monoplanar collet insert 370 into the monoplanar receiver 300 to form the monoplanar receiver sub-assembly 32 in the shipping state configuration is shown in FIGS. 78-86. The method of pre-assembling the monoplanar receiver sub-assembly 32 can be substantially similar to the pre-assembly methods for the multiplanar embodiments described above, with the exception of changes necessitated by the different structures of the monoplanar components. For instance, as shown in FIGS. 79-80, the monoplanar cap retainer 350 can be downloaded at an angle through the central bore 320 of the monoplanar receiver 300 until the lower opposite peg 366 reaches the level of and enters into the extra space provided by one of the opposed expansion recesses 330. This can provide the extra spaced need to re-aligned and level the monoplanar cap retainer 350 within the cavity 334 of the monoplanar receiver 300 as the monoplanar cap retainer 350 is lowered into engagement with the spherical seating surface 338 and upward-facing pivot grooves 337 of the monoplanar receiver 300.

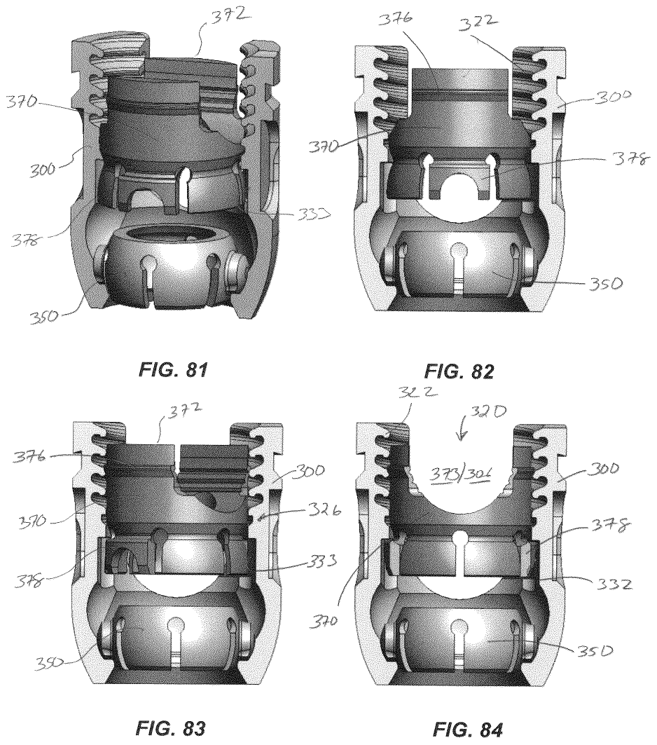

With reference to FIGS. 81-86, the monoplanar collet insert 370 can then be downloaded into the central bore 320 of the monoplanar receiver 300, with the insert arms 372 aligned with the rod channel 306, until the opposite lower flange fingers 378 reach the level of the access recesses 333 (FIGS. 81-82). The monoplanar collet insert 370 can then be rotated around the vertical centerline axis of the monoplanar receiver 300 so that the opposite lower flange fingers 378 begin to enter the horizontal access recesses 333, as shown in FIG. 83. The rotation of the monoplanar collet insert 370 can continue for a full 90 degrees or quarter turn, until the insert channel 373 becomes aligned with the rod channel 306 of the monoplanar receiver 300 and the opposite lower flange fingers 378 completely slide into the opposed vertical side pockets 332 of the central bore 320, as shown in FIG. 84.

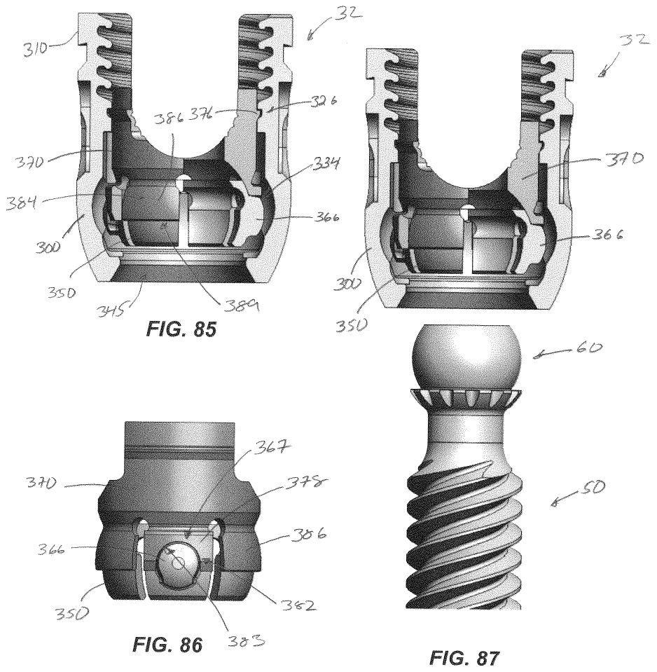

With the opposite upper outer ridges 376 being located in the region of the discontinuous guide and advancement structure 322 of the upright arms 310, the monoplanar collet insert 370 can then be pushed further downward vertically within the central bore 320 until opposite upper outer ridges 376 snap into the upper shipping state grooves 326 of the upright arms 310, as shown in FIG. 85. Also shown in FIG. 85, the monoplanar cap retainer 350 can then be uploaded through the lower opening of the lower collet portion 384 defined by the discontinuous annular bottom surface 389, with the curvate collet fingers 386 flexing open within the expansion portion of the internal cavity 334 of the monoplanar receiver 300, until the cap retainer 350 is captured by the lower collet portion 384 in a stabilized position that is suspended and centered over the bottom opening 345. Also during the process of uploading the monoplanar cap retainer 350 into the lower collet portion 384, the opposite pegs 366 can enter the circular cutouts 382 of the opposite lower flange fingers 378 until the partial circular cylindrical upper surfaces 367 of the monoplanar cap retainer 350 slidably engage with the downward-facing partial cylindrical surfaces 383 of the opposite lower flange fingers 378, as shown in the isolated side view the monoplanar cap retainer 350 and the monoplanar collet insert 370 of FIG. 86. The downward displacement of the monoplanar collet insert 370 into engagement with the upper shipping state grooves 326 the monoplanar receiver 300 and the uploading of the monoplanar cap retainer 350 into the lower collet portion 384, as shown in the drawings, can comprise the final steps for pre-assembling the monoplanar receiver sub-assembly 32 of the monoplanar bone anchor assembly.

With reference to FIGS. 87-91, the monoplanar receiver sub-assembly 32 can now be assembled with the bi-spheric shank head 60 of the bone anchor or shank 50. The method of assembling the monoplanar receiver sub-assembly 32 with the bi-spheric shank head 60 can be substantially similar to the assembly methods for the multiplanar embodiments described above, with the addition that the opposite pegs 366 projecting outwardly from opposite sides of the discontinuous outer spherical surface 356 of the monoplanar cap retainer 350 can be pushed outward into the opposed expansion recesses 330 of the monoplanar receiver 300 as the collet fingers 364 of the cap retainer 350 expand toward their point of maximum expansion, as shown in FIG. 89.

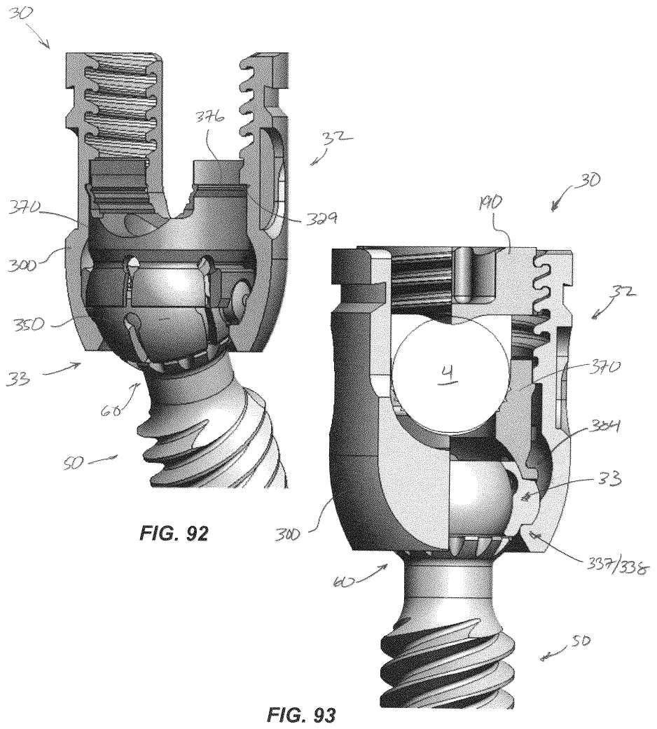

Once the monoplanar shank head sub-assembly 33 has been assembled and secured within the lower collet portion 384 (FIG. 90), the monoplanar collet insert 370 can then be downwardly deployed with a deployment tool (not shown) until the lower portion of the discontinuous outer spherical surface 356 of the monoplanar cap retainer 350 becomes engaged within the spherical seating surface 138 of the monoplanar receiver 100 and the opposite pegs 366 become engaged within the upward-facing pivot grooves 337, respectively, at the same time that the opposite upper outer ridges 376 snap below the downward-facing upper arcuate planar surfaces 329 (FIGS. 91-92). As described above, in one aspect the pivoting monoplanar shank head sub-assembly 33 (i.e. the monoplanar cap retainer 350 the bi-spheric shank head 60) can be secured against the downwardly-opening concave inner spherical surface 388 of the monoplanar collet insert 370 and against the spherical seating surface 338 and pivot grooves 337 of the monoplanar receiver 300 with a non-floppy frictional engagement, or pre-lock friction fit that allows for the bone anchor or shank 50 to pivot in a single plane relative to the monoplanar receiver 300 with some resistance (FIG. 92). The coupling of the universal bi-spheric shank head 60 of the bone anchor or shank 50 with the monoplanar receiver sub-assembly 32 can complete the formation of the monoplanar bone anchor assembly 30 in its initial configuration, one in which the monoplanar bone anchor assembly 30 is ready to be implanted into the vertebrae of a patient or to receive the elongate rod and the closure.

Illustrated in FIG. 93 is the monoplanar bone anchor assembly 30 after final assembly with the elongate rod 4 and the single piece closure 190. As with the multiplanar embodiments, in this configuration the monoplanar collet insert 370 can be pressed further downward within the central bore of the monoplanar receiver 300 by the elongate rod 4 and closure 190 until a hard or full frictional lock between the monoplanar shank head sub-assembly 33, the lower collet portion 384 of the monoplanar collet insert 370, and the spherical seating surface 338 and pivot grooves 337 of the monoplanar receiver 300 is achieved, thereby preventing further movement between the monoplanar shank head sub-assembly 33 and the monoplanar receiver 300. Moreover, even though the fully assembled and locked monoplanar bone anchor assembly 30 is only illustrated with a 6.0 mm diameter rod 4 in FIG. 93, it will be appreciated that the upward-facing insert channel 373 of the monoplanar collet insert 370 can also accommodate the smaller 5.5 mm diameter rod, similar to the other embodiment of the collet insert described above.

### Monoaxial Bone Anchor Assembly

FIG. 94 is an exploded perspective view of one representative embodiment of the monoaxial or ‘non-pivotal but rotatable’ bone anchor assembly 40 illustrated in FIG. 1(e) that is configured, as noted above, to substantially eliminate pivotal motion of the bone anchor relative to the receiver sub-assembly (except perhaps for a slight toggle) while still providing for a 360-degree range of rotational motion around the longitudinal axis 51 of the bone anchor 50. The monoaxial bone anchor assembly 40 can include the same bone anchor or shank 50 described above, having a bi-spheric shank head 60 and an anchor portion 84 opposite the bi-spheric shank head 60 for securement or attachment to the bone of a patient.

Similar to the multiplanar and monoplanar bone anchor assemblies discussed above, the monoaxial bone anchor assembly 40 can also include the monoaxial receiver 400 that can be initially non-pivotably secured to the bi-spheric shank head 60 with a number of separate internal components that have been pre-assembled into the central bore 120 and the rod channel 106 to form the monoaxial receiver sub-assembly 42. These internal components can include, but are not limited to, a monoaxial cap retainer 450 and a monoaxial collet insert 470. After the elongate rod 4 has been positioned within the lower portion of the rod channel of the monoaxial receiver, the same single-piece closure 190 (or another appropriate type of closure) can be threadably or otherwise secured into an upper portion of the rod channel to apply pressure to an upper surface of the elongate rod, thereby locking both the elongate rod and the monoaxial bone anchor assembly 40 into a final locked position.

Differences between monoaxial bone anchor assembly 40 of FIG. 94 and the multiplanar bone anchor assemblies described above can include the replacement of the multiplanar receiver with a monoaxial receiver 400, as shown in FIGS. 95-96. The monoaxial receiver 400 can have many of the same features as the multiplanar version, with the addition of an enlarged spherical seating surface 438 and slightly reconfigured side pockets 432 and access recesses 433.

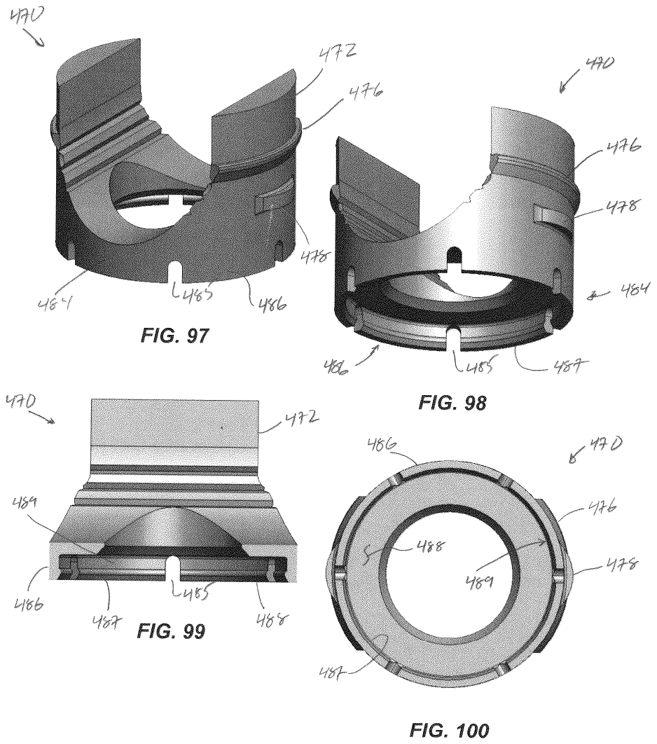

With reference to FIGS. 97-100, the additional differences can also include the replacement of the multiplanar versions of the collet insert with a monoaxial collet insert 470. The monoaxial collet insert 470 can have many of the same features as the multiplanar version, but with changes to the lower collet portion 484 that includes collet fingers 486, separated by vertical slots 485, that have been shortened and shaped to define a discontinuous inwardly-projecting circular ridge 487 that, in turn, defines a discontinuous lower circumferential recess 489. The changes can also include the replacement of the lower concave inner surface with an annular planar lower surface 488 configured to engage with the top surface of the monoaxial cap retainer 450, as described below.

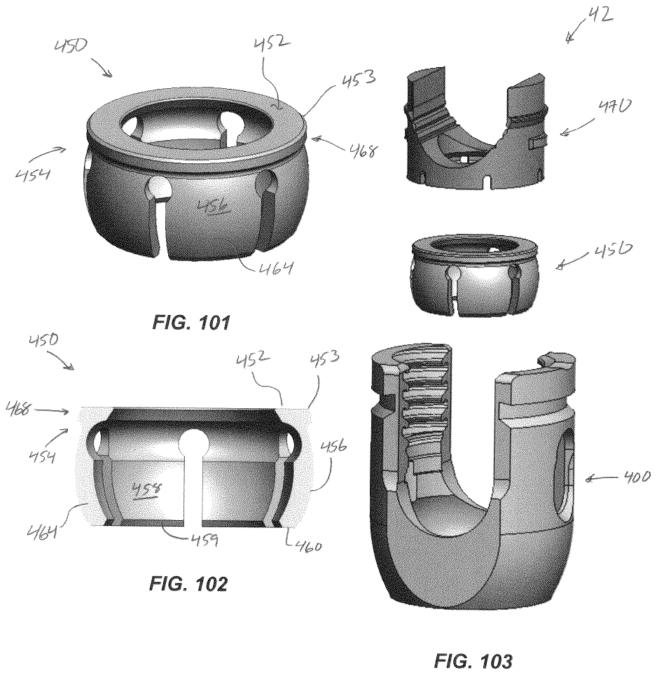

With reference to FIGS. 101-102, the additional differences can also include the replacement of the multiplanar cap retainer with a monoaxial cap retainer 450. The monoaxial cap retainer 450 can have many of the same features as the multiplanar version, with changes to the solid or continuous upper ring portion 454 that includes an outwardly-projecting circular flange 468 that partially defines the annular planar top surface 452, and which extends the continuous circular outer edge 453 of the annular planar top surface 452 radially outward to increase the width of the annular planar top surface 452.

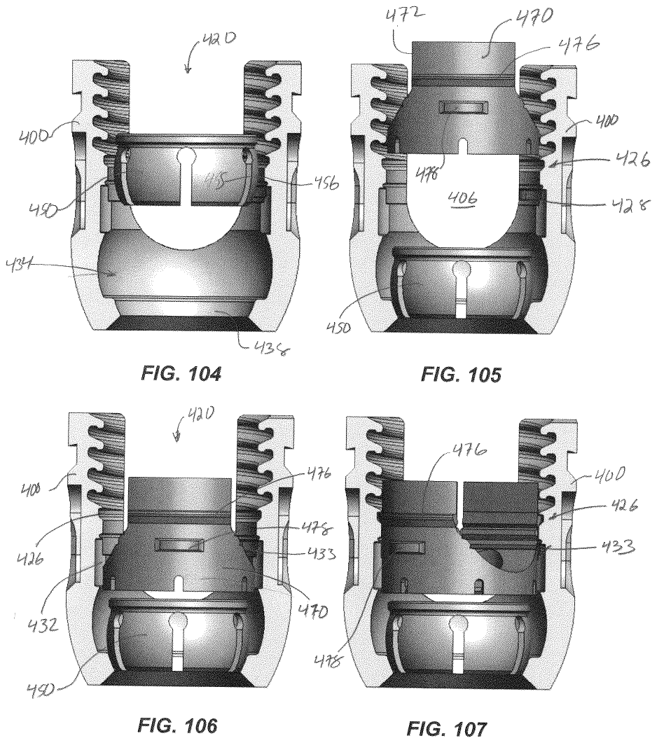

The pre-assembly of the monoaxial cap retainer 450 and monoaxial collet insert 470 into the monoaxial receiver 400 to form the monoaxial receiver sub-assembly 32 in the shipping state configuration is shown in FIGS. 103-110. The method of pre-assembling the monoaxial receiver sub-assembly 42 can be substantially similar to the pre-assembly methods for the multiplanar embodiments described above. For instance, as shown in FIGS. 104-105, the monoaxial cap retainer 450 can be downloaded through the central bore 420 until the discontinuous outer spherical surface 456 engages with the spherical seating surface 438 at the lower end of the cavity 434 of the monoaxial receiver 400.

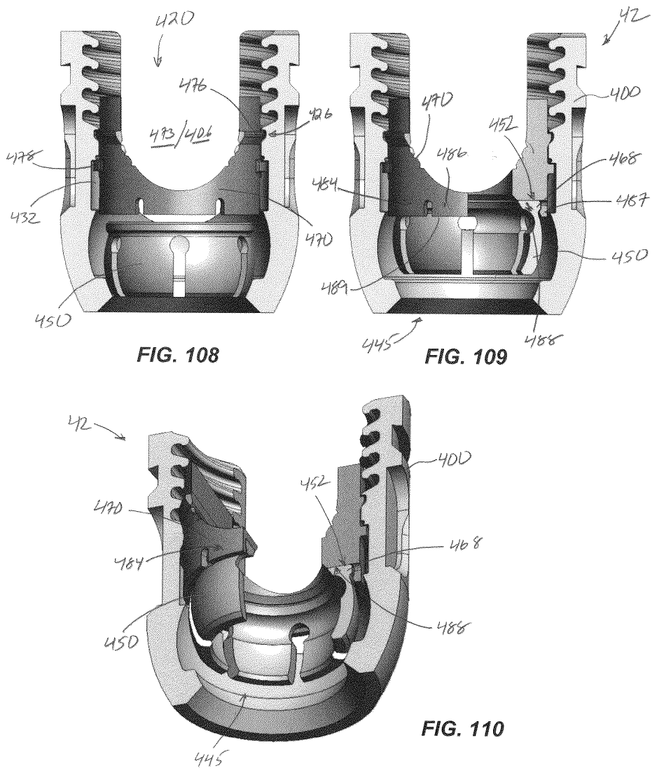

After the monoaxial cap retainer 450 is seated on the spherical seating surface 438 of the monoaxial receiver 400, the monoaxial collet insert 470 may then be top-loaded or down-loaded into the central bore 420 with the insert arms 472 being aligned with the rod channel 406 (FIG. 105) until the opposite upper outer ridges 476 reach the level of the opposed shipping state grooves 426 (formed into the rod channel portion of the central bore 120 for this type of twist-in-place deployable collet insert 470) and the outwardly-projecting indexing nubs 478 reach the level of the horizontal access recesses 433 (FIG. 106). The monoaxial collet insert 470 can then be rotated around the vertical centerline axis of the monoaxial receiver 400 so that the leading edges of the opposite upper outer ridges 476 begin to enter into the upper shipping state grooves 426 and the outwardly-projecting indexing nubs 478 enter the horizontal access recesses 433, as shown in FIG. 107. The rotation of the monoaxial collet insert 470 can continue for a full 90 degrees or quarter turn, until the insert channel 473 becomes aligned with the rod channel 406 of the monoaxial receiver 400 and the indexing nubs 428 completely slide into the opposed vertical side pockets 432 of the central bore 420, as shown in FIG. 108.

With reference to FIGS. 109-110, the monoaxial cap retainer 450 can then be uploaded through the lower opening of the lower collet portion 484 defined by the discontinuous annular bottom surface 489, with the collet fingers 486 flexing open within the central bore 420 of the monoaxial receiver 400, until the discontinuous inwardly-projecting circular ridge 487 of the lower collet portion 484 can snap under the outwardly-projecting circular flange 468 simultaneous with the annular planar top surface 452 of the monoaxial cap retainer 450 engaging the annular planar lower surface 488 of the monoaxial collet insert 470, effectively securing the two components together and preventing the monoaxial cap retainer 450 from pivoting relative to the monoaxial collet insert 470. The full rotation of the monoaxial collet insert 470 into its initial position within the monoaxial receiver 400 and the uploading of the monoaxial cap retainer 450 into the lower collet portion 484, as shown in the drawings, can comprise the final steps for pre-assembling the monoaxial receiver sub-assembly 42 of the monoaxial bone anchor assembly.

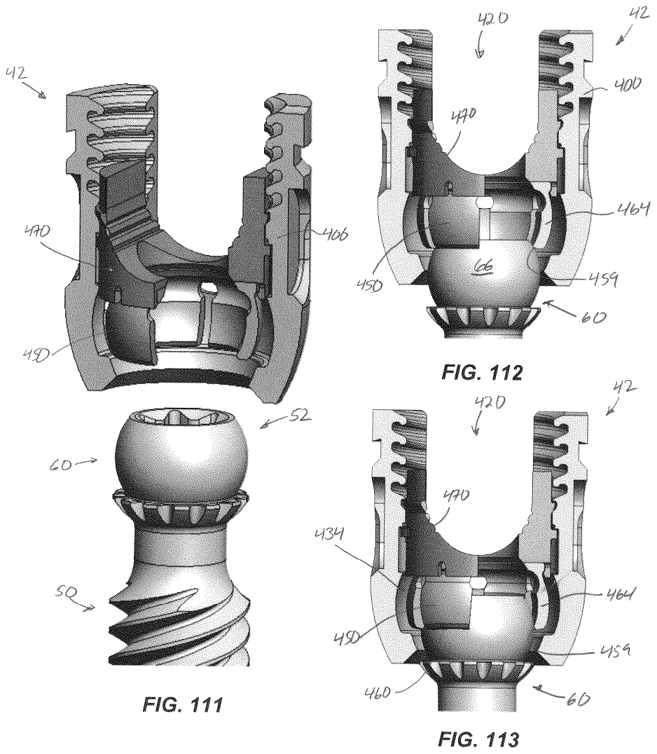

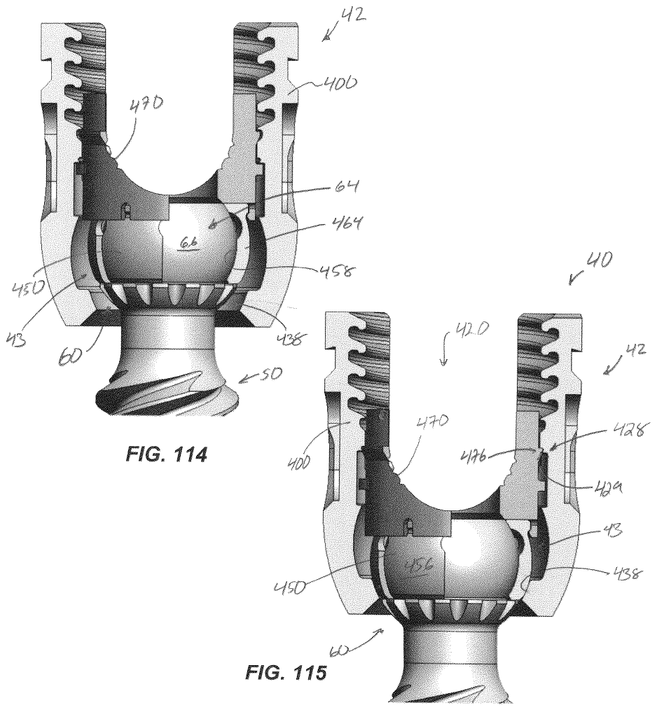

With reference to FIGS. 111-115, the monoaxial receiver sub-assembly 42 can now be assembled with the bi-spheric shank head 60 of the bone anchor or shank 50. The method of assembling the monoaxial receiver sub-assembly 42 with the bi-spheric shank head 60 can be substantially similar to the assembly methods for the multiplanar embodiments described above. In particular, the monoaxial receiver sub-assembly 42 can first be positioned above the proximal end 52 of the bone anchor 50 (FIG. 111) and then dropped downward (or the bone anchor moved upward, depending on the frame of reference of the reader) until the upper spherical surface 66 of the bi-spheric shank head engages the beveled edge surfaces 459 of the collet fingers 464 of the monoaxial cap retainer 450 (FIG. 112). The monoaxial receiver sub-assembly 42 can continue to move downward as the bi-spheric shank head 60 pushes against the beveled edge surfaces 459 to expand the collet fingers 464 of the monoaxial cap retainer 450 within the upper expansion portion of the internal cavity 434, until the discontinuous annular bottom surface 460 reaches the level of the hemisphere plane of the bi-spheric shank head 60 and the collet fingers 464 of the monoaxial cap retainer 450 are at their point of maximum expansion (FIG. 113). The monoaxial receiver sub-assembly 42 can continue to move downward until the upper partial spherical portion 64 of the bi-spheric shank head 60 becomes fully captured by the monoaxial cap retainer 450 to create a non-articulating monoaxial shank head sub-assembly 43 (FIG. 114).

Once the monoaxial shank head sub-assembly 43 has been assembled and secured within the lower collet portion 484, the monoaxial collet insert 470 can then be downwardly deployed with a deployment tool (not shown) until the lower portion of the discontinuous outer spherical surface 456 of the monoaxial cap retainer 450 becomes engaged within the spherical seating surface 438 of the monoaxial receiver 400 at the same time that the opposite upper outer ridges 476 snap below the downward-facing upper arcuate planar surfaces 429 of the lower locking groove 428 (FIG. 115). In one aspect the monoaxial collet insert 470, monoaxial cap retainer 450 and capture structure 60 can be downwardly deployed to a friction fit configuration that allows for relative frictional rotation between the capture structure 60 and the monoaxial cap retainer 450 or between the monoaxial cap retainer 450 and the collet insert 470, while preventing pivoting and lateral motions between the components. The coupling of the universal bi-spheric shank head 60 of the bone anchor or shank 50 with the monoaxial receiver sub-assembly 42 can complete the formation of the monoaxial bone anchor assembly 40 in its initial configuration, one in which the monoaxial bone anchor assembly 40 is ready to be implanted into the vertebrae of a patient or to receive the elongate rod and the closure.

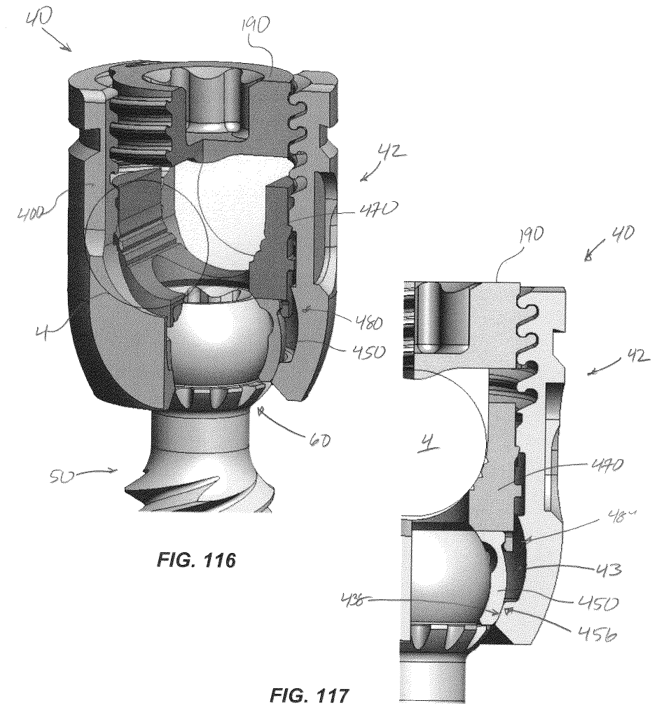

Illustrated in FIGS. 116-117 is the monoaxial bone anchor assembly 40 after final assembly with the elongate rod 4 and the single piece closure 190. As with the multiplanar embodiments, in this configuration the monoaxial collet insert 470 can be pressed further downward within the central bore of the monoaxial receiver 400 by the elongate rod 4 and closure 190 until a hard or full frictional lock between the monoaxial shank head sub-assembly 43, the lower collet portion 484 of the monoaxial collet insert 470, and the spherical seating surface 438 of the monoaxial receiver 400 is achieved, thereby preventing further movement between the monoaxial shank head sub-assembly 43 and the monoaxial receiver 400. Moreover, even though the fully assembled and locked monoaxial bone anchor assembly 40 is only illustrated with a 6.0 mm diameter rod 4 in the drawing figures, it will be appreciated that the upward-facing insert channel 473 of the monoplanar collet insert 470 can also accommodate the smaller 5.5 mm diameter rod, similar to the other embodiment of the collet insert described above.

### Multiplanar Bone Anchor Assembly With Grooved Cap Retainer

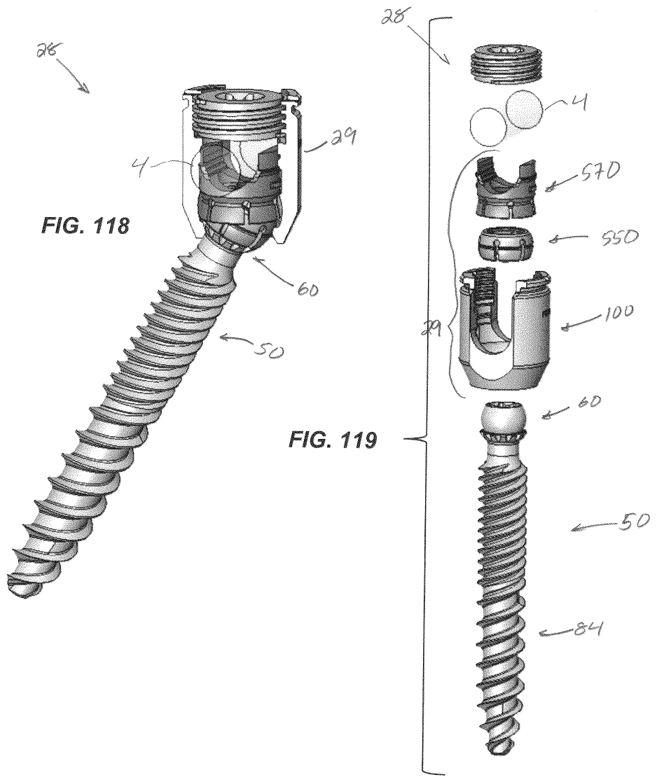

FIG. 118 is partially-sectioned perspective view of another representative embodiment of the multiplanar bone anchor assembly 28, with the multiplanar receiver sub-assembly 29 and an elongate rod being connected to the bi-spheric shank head 60 of the bone anchor 50 and with the bone anchor 50 being pivoted and locked at an angle with respect to the receiver of the multiplanar receiver sub-assembly 29. FIG. 119 is an exploded perspective view of the same multiplanar bone anchor assembly 28 and rod 4.

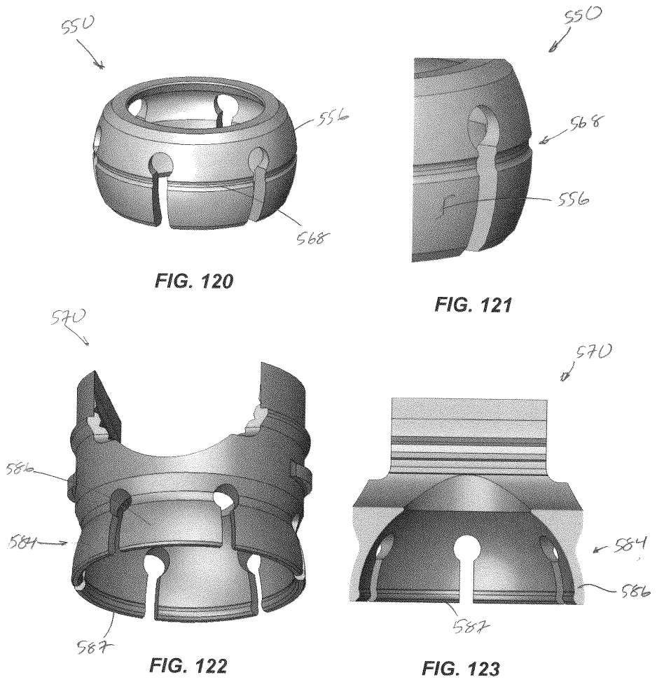

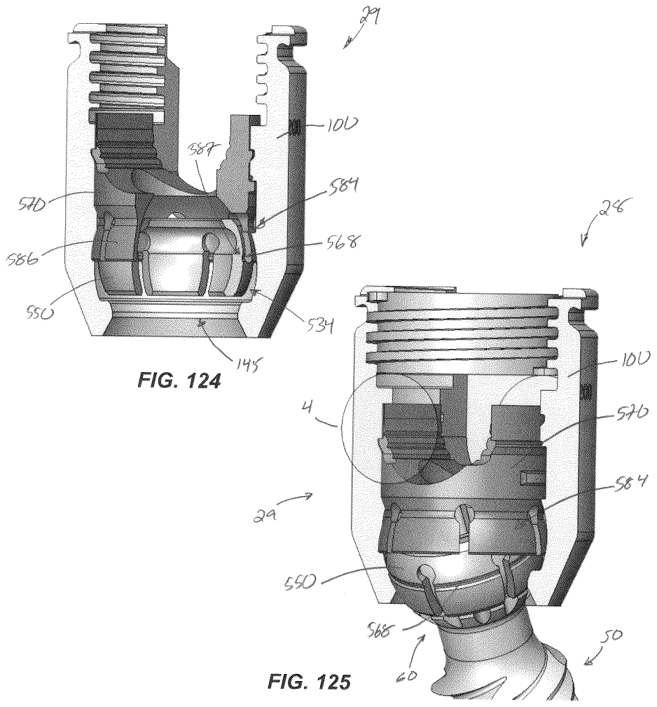

With continued reference to FIGS. 120-123, the multiplanar bone anchor assembly 28 differs from the similar embodiments described above in that the multiplanar cap retainer 550 can be modified to include a discontinuous horizontal groove 568 formed into and extending circumferentially around the discontinuous outer spherical surface 556 of the multiplanar cap retainer 550, at about the midline or equator of the partially spherical component. The multiplanar collet insert 570 can also differ in that it includes a discontinuous, inwardly-protruding lower ridge 587 at the bottom edges of the collet fingers 586 that is complementary with the horizontal groove 568, so as to enter the horizontal groove 568 upon the pre-assembly of the multiplanar cap retainer 550 and the multiplanar collet insert 570 into the multiplanar receiver 100 to form the multiplanar receiver sub-assembly 29 in the shipping state configuration, as shown in FIG. 124. In one aspect the circular tongue-and-groove type engagement between the discontinuous horizontal groove 568 of the cap retainer 550 and the discontinuous, inwardly-protruding lower ridge 587 of the collet portion 584 of the collet insert 570 can serve to better hold the cap retainer 550 in the stabilized and centralized position within the internal cavity 534 above the bottom opening 145, so as to prevent the cap retainer 550 from shifting or pivoting or otherwise allowing the center aperture of the cap retainer 550 to become mis-aligned relative to the bottom opening 145 of the receiver 100 in a way that would hinder or prevent the uploading of the bi-spheric shank head 60 into the multi-planar receiver sub-assembly 29.

Once the bi-spheric shank head or capture structure 60 has been uploaded through the bottom opening and into the multiplanar cap retainer 550, the multiplanar collet insert 570, cap retainer 550 and capture structure 60 can be downwardly deployed with a tool or tooling (not shown) within the multiplanar receiver 100 to the friction fit configuration. This can provide for relative frictional pivotal and rotational movement between the cap retainer 550 and the collet insert 570 and receiver 100 until the elongate rod is positioned within the channel and the pivotal bone anchor assembly 28 is locked with the closure, as shown in FIG. 125.

As indicated above, the spinal fixation system and bone anchor assemblies of the present disclosure have been described herein in terms of representative embodiments and methodologies considered by the inventors to represent best modes of carrying out the one or more inventions disclosed herein. It will be understood by the skilled artisan, however, that a wide range of additions, deletions, and modifications, both subtle and gross, may be made to the illustrated embodiments of the pivotal and non-pivotal bone anchor assemblies, to the modular spinal fixation system, and to the representative type of bi-spheric shank head, and that these and other revisions might be made by those of skill in the art without departing from the spirit and scope of the one or more inventions that are to be constrained only by their respective claims.

What is claimed is:

1. A bone anchor assembly for securing an elongate rod to a bone of a patient with tooling, the bone anchor system comprising:
a bone anchor comprising a longitudinal axis, a shank head at a proximal end, and an anchor portion opposite the shank head configured for fixation to the bone, the shank head including:
an upper partial spherical portion comprising an annular planar top surface at an upper end of the shank head and an upper spherical surface having a first diameter extending downward from the planar top surface past a hemisphere plane to a circular inner edge of an upward-facing ledge; and
a lower partial spherical portion comprising a lower spherical surface having second diameter greater than the first diameter extending downward from a circular outer edge of the upward-facing ledge toward a neck portion that connects the shank head to the anchor portion;
a receiver having a vertical centerline axis, an upper portion defining a receiver channel configured to receive the elongate rod, and a base defining a lower portion of a central bore formed around the vertical centerline axis and communicating with a bottom surface of the receiver through a bottom opening, the central bore extending upwardly from the bottom opening through the channel to a top of the receiver and including a guide and advancement structure mateable with a closure proximate the top of the receiver, opposed upper inner engagement grooves below the guide and advancement structure, and a spherical seating surface adjacent the bottom opening;
a cap retainer positionable within the lower portion of the central bore having a discontinuous outer spherical surface configured to engage the spherical seating surface of the receiver and a plurality of retainer collet fingers separated by vertically-extending slots configured to resiliently expand to receive and capture the upper partial spherical portion of the shank head within the receiver when the shank head is uploaded through the bottom opening; and
a collet insert having an upper insert portion defining an insert channel configured to engage the elongate rod and opposite outer engagement ridges extending radially outward from the upper insert portion, and a lower collet portion comprising a plurality of insert collet fingers separated by vertically-extending slots and configured to resiliently expand to receive and capture the cap retainer,
wherein the collet insert is positionable within the central bore above the cap retainer with the opposite outer engagement ridges positioned within the opposed upper inner engagement grooves to maintain an initial vertical position of the collet insert within the central bore, and
wherein prior to uploading the shank head through the bottom opening, the cap retainer is uploadable into the lower collet portion of the collet insert with a lower opening of the cap retainer spaced above the bottom opening of the receiver.

2. The bone anchor assembly of claim 1, further comprising a discontinuous horizontal groove formed into and extending circumferentially around the discontinuous outer spherical surface of the cap retainer and a discontinuous lower ridge located at bottom edges of the insert collet fingers,
wherein the discontinuous lower ridge is positionable within the a discontinuous horizontal groove to further secure the cap retainer within the lower collet portion of the collet insert.

3. The bone anchor assembly of claim 1, wherein the shank head further comprises an internal drive structure surrounded by the annular planar top surface and extending downward from the upper end of the shank head and configured to mate with a drive tool.

4. The bone anchor assembly of claim 3, wherein the bone anchor further comprises a shank body having an axial bore extending from the internal drive structure down to a distal end of the anchor portion and configured to receive a guide wire, the anchor portion of the shank body being configured for implantation in the bone about the guide wire with the drive tool prior to the shank head being uploaded into the central bore of the receiver.

5. The bone anchor assembly of claim 1, wherein a discontinuous annular bottom surface of the cap retainer is configured to engage an upper ledge surface of the upward-facing ledge to align the cap retainer to the shank head when capturing the shank head within the central bore of the receiver.

6. The bone anchor assembly of claim 5, wherein a diameter of the discontinuous outer spherical surface of the cap retainer is substantially equal to the second diameter of the lower spherical surface of the shank head to form a single diameter, articulating shank head sub-assembly having the second diameter that is greater than the diameter of the bottom opening of the receiver upon capturing the shank head within the central bore of the receiver.

7. The bone anchor assembly of claim 5, wherein the cap retainer includes an annular planar upper surface that alignable flush with the annular planar top surface of the shank head upon capturing the shank head within the central bore of the receiver.

8. The bone anchor assembly of claim 5, wherein the discontinuous annular bottom surface of the cap retainer and the upper ledge surface of the upward-facing ledge are substantially planar surfaces extending perpendicular to the longitudinal axis of the bone anchor.

9. The bone anchor assembly of claim 1, wherein the upward-facing ledge and the lower partial spherical portion of the shank head include a plurality of open, vertically aligned flutes arranged circumferentially around the shank head and extending downwardly through and below the upward-facing ledge.

10. The bone anchor assembly of claim 1,
wherein the collet insert further comprises a pair of insert arms extending upward from a circular center portion to define the insert channel with the opposite outer engagement ridges extend radially outward from outer side surfaces of the insert arms, and
wherein the opposite outer engagement ridges are configured to rotate into the opposed upper inner engagement grooves of the central bore upon rotation of the collet insert about the vertical centerline axis of the receiver to define the initial vertical position of the collet insert in the central bore.

11. The bone anchor assembly of claim 1, wherein the shank head is configured for axial rotation about the longitudinal axis of the shank relative to the receiver prior to locking the bone anchor assembly with the closure.

12. The bone anchor assembly of claim 1, wherein after the shank head is uploaded into the lower collet portion of the collet insert, the collet insert, cap retainer and shank head are downwardly deployable together within the central bore with the tooling until the discontinuous outer spherical surface engages the spherical seating surface of the receiver.

13. The bone anchor assembly of claim 12, wherein upon the downward deployment of the collet insert with tooling, the opposite outer engagement ridges are configured to snap into opposed inner locking grooves formed into the central bore of the receiver to prevent the collet insert from moving back up within the receiver.
